(19) United States
(12) Patent Application Publication
(10) Pub. No.: US 2025/0332449 A1
(43) Pub. Date: Oct. 30, 2025

(54) REAL-TIME VERIFICATION OF RADIOTHERAPY DELIVERY SYSTEM OPERATION

(71) Applicant: RefleXion Medical, Inc., Hayward, CA (US)

(72) Inventors: Mingshan SUN, Hayward, CA (US); George Andrew ZDASIUK, Hayward, CA (US)

(21) Appl. No.: 19/259,997

(22) Filed: Jul. 3, 2025

Related U.S. Application Data
(63) Continuation of application No. PCT/US2023/085347, filed on Dec. 21, 2023.
(60) Provisional application No. 63/478,572, filed on Jan. 5, 2023.
(60) Provisional application No. 63/505,865, filed on Jun. 2, 2023.

(51) Int. Cl. A61N 5/10

(52) U.S. Cl. CPC A61N 5/1071; A61N 5/1039; A61N 5/1045; A61N 5/1075; A61N 5/1077; A61N 2005/1074

(57) ABSTRACT

Disclosed herein are methods for verifying whether a radiotherapy delivery system is delivering therapeutic radiation to the target region in accordance with instructions from the radiotherapy system controller. The methods utilize imaging data acquired by a radiation imager (e.g., MV detector) that is located across from (e.g., opposite) the therapeutic radiation source. In some variations, the imaging data from the radiation imager is used to determine whether the components of the radiation beam-shaping assembly are in the location specified by the controller instructions. Also disclosed herein are methods for verifying whether the radiation beams emitted by the therapeutic radiation source intersect with a target region and/or a contour around the target region, including methods for determining delivered fluence and dose estimates.

### CROSS-REFERENCE TO RELATED APPLICATIONS

This application is a continuation of International Application No. PCT/US2023/085347 which claims priority to U.S. Provisional Patent Application Ser. No. 63/478,572 filed Jan. 5, 2023, and U.S. Provisional Patent Application Ser. No. 63/505,865 filed Jun. 2, 2023, the disclosures of which are hereby incorporated by reference in their entirety.

### BACKGROUND

External beam radiotherapy is a cancer treatment modality that involves the emission of radiation to a patient and aims to deliver a lethal dose of radiation to a tumor while sparing healthy tissue. Radiotherapy delivery systems have a therapeutic radiation source that can be moved around a patient couch and radiation beam-shaping components in the radiation beam path of the therapeutic radiation source. The beam-shaping components (e.g., jaws, collimators) may have movable radiation-blocking elements that can be adjusted to focus and shape the radiation beam to target the tumor and avoid surrounding anatomy. For example, a multi-leaf collimator (MLC) may have a plurality of independently movable leaves that can each be positioned to shape the radiation beam as desired. Radiotherapy systems also have a radiation detector (which may also be referred to as a radiation imager) located opposite the therapeutic radiation source to take measurements of the radiation emitted by the therapeutic radiation source. During a treatment session, a radiotherapy delivery system is used to deliver a prescribed dose of radiation to a tumor by emitting radiation shaped by the beam-shaping components from multiple firing positions around the patient. In most radiotherapy delivery systems, the instructions for delivering a prescribed dose to a patient are generated as part of treatment planning and once the radiotherapy delivery system completes the execution of the instructions, the treatment session ends.

However, it can be difficult to confirm whether the radiation emitted to the patient during a treatment session is consonant with the planned radiation dose. While the radiation detector can take measurements of the radiation emitted by the therapeutic radiation source, some of the radiation emitted to the patient is absorbed while some of the radiation may be scattered, which can lead to inaccurate or ambiguous measurements on the radiation detector. Additional discrepancies between the emitted radiation and planned radiation can result if there are radiotherapy delivery system component errors or deviations from the treatment plan machine instructions. In situations where there is an unacceptable difference between the emitted radiation and the planned radiation, the treatment session may be paused or stopped to help prevent the unintentional irradiation of healthy tissue. Accordingly, for the safety of the patient, it is desirable to develop methods to determine the quantity and location of the radiation dose delivered during a treatment session.

### SUMMARY

Disclosed herein are methods for verifying whether a radiotherapy delivery system is delivering therapeutic radiation to the target region in accordance with instructions from the radiotherapy system controller. Also disclosed herein are methods for determining whether therapeutic radiation is directed to the desired region of the patient, for example, within the contours of a region of interest (i.e., any clinician-defined region of a patient and/or phantom). The methods may utilize imaging data acquired by a radiation imager (e.g., MV detector) that is located across from (e.g., opposite) the therapeutic radiation source. In some variations, the imaging data from the radiation imager may be used to determine whether the components of the beam-shaping assembly (e.g., multi-leaf collimator or MLC) are in the location specified by the controller instructions. Also disclosed herein are methods for verifying whether the radiation beams emitted by the therapeutic radiation source intersect with a target region and/or a contour of a target region. In some variations, the contour around the target region may encompass the motion range of the target region, and/or margins for uncertainty in the location of the target region, and may be, for example, a planning target volume (PTV), internal target volume (ITV), biology-tracking zone (BTZ), and the like.

One variation of a method for verifying the functionality of the multi-leaf collimator (MLC) of a radiotherapy system during a radiation delivery session may include generating a MLC pattern during the radiation delivery session, wherein the MLC pattern designates an expected MLC leaf location for each leaf of the MLC, wherein the MLC is located in front of a radiation source, emitting radiation from a radiation source, wherein the emitted radiation is shaped by the MLC, acquiring a radiation measurement of the emitted radiation using a radiation imager located opposite the radiation source, determining a location of an MLC leaf using the radiation measurement, comparing the determined MLC leaf location with the expected MLC leaf location, and generating an MLC leaf location notification. Some variations may further comprise calculating a time duration of the MLC leaf at the determined MLC leaf location using the radiation measurement, comparing the calculated time duration with an expected time duration of the MLC leaf at the expected location, and generating an MLC dwell time notification based on the comparison between the calculated time duration and the expected time duration. Additionally, or alternatively, some methods may include calculating an intensity of the emitted radiation using the radiation measurement, comparing the calculated intensity with an expected radiation intensity, and generating an intensity notification based on the comparison between the calculated intensity and the expected radiation intensity. The emitted radiation may include one or more radiation pulses and the expected radiation intensity comprises an expected number of radiation pulses, and calculating the intensity of the emitted radiation may include calculating a number of radiation pulses using the radiation measurement. In some variations, comparing the calculated intensity with the expected radiation intensity may include comparing the calculated number of radiation pulses with the expected number of radiation pulses. In some variations, no patient is present when the radiation is emitted while in other variations, a patient is present when the radiation is emitted. The radiation measurement may include intensity values of each pixel of the radiation imager, and determining a location of the MLC leaf may include identifying pixels of the radiation imager that had an intensity over a threshold level and mapping the identified pixels to the MLC leaf and its location. The location of the MLC leaf may be determined based on a pattern of pixels having intensity values below the threshold level and/or the location of the MLC leaf may be determined based on a pattern of pixels having intensity values at or above the threshold level. Optionally, the radiation measurement may include one or more frames where each frame has intensity values of each pixel of the radiation imager over a pre-determined acquisition time period, and determining the location of the MLC leaf may further include calculating a dwell time that the MLC leaf is at the determined location based on a number of frames in which a set of pixels have an intensity over the threshold value. In some variations, the method may further include comparing the calculated dwell time with an expected dwell time, and generating a notification may further include generating a notification if the calculated dwell time is different from the expected dwell time.

Another variation of a method for verifying radiation beamlet delivery to a region of interest may include emitting a radiation beamlet from a radiation source, where the emitted radiation beamlet is defined by an opening of a MLC located in front of the radiation source, acquiring a radiation measurement of the emitted radiation beamlet using a radiation imager located opposite the radiation source, determining a path of the radiation beamlet from the radiation source to the radiation imager using the radiation measurement, and generating a graphical representation that includes the radiation beamlet path and a location of a region of interest. The method may further include generating a notification if the radiation beamlet path does not intersect the region of interest. The region of interest may be defined by a boundary and the method may include generating a notification if the radiation beamlet path does not cross the boundary of the region of interest. The region of interest may include an internal tumor volume (ITV) and/or a biology tracking zone (BTZ). In some variations, no patient is present when the radiation is emitted while in other variations, a patient is present when the radiation is emitted. Determining the path of the radiation beamlet may include identifying a first location of the MLC opening, identifying a second location on the radiation imager based on the acquired radiation measurement, and determining the radiation beamlet path by defining a line between the first location and the second location. In some variations, the graphical representation may include an outline of a boundary of the region of interest and a line that represents the radiation beamlet path.

Also described herein is a variation of a method for verifying a delivered fluence, which may include emitting radiation beamlets from a radiation source, generating a delivered radiation intensity map by forward-projecting the emitted radiation beamlets, where the radiation intensity map comprises a spatial plot of radiation intensities, defining a high-fluence contour that encompasses a region of the delivered radiation intensity map that has intensity levels at or above a radiation intensity threshold, determining a contour difference between the high-fluence contour and a treatment planning contour by registering the treatment planning contour with the delivered radiation intensity map, and generating a graphical representation that includes the high-fluence contour and the treatment planning contour overlaid on the delivered radiation intensity threshold, and a notification of the contour difference. The emitted radiation beamlets may include an irradiation field and generating the delivered radiation intensity map may include calculating emitted fluences based on the openings of the MLC, radiation source pulses, and radiation source locations, and plotting the emitted fluences over the irradiation field. The radiation intensity map may be a 3D spatial plot and/or a 2D spatial plot. In some variations, defining a high-fluence contour may include identifying a pixel of the radiation intensity map having a higher intensity value than the other pixels in the radiation intensity map, calculating a threshold intensity value by calculating a percentage of the highest intensity value, identifying a group of pixels in the radiation intensity map that have intensity values at or above the threshold intensity value, and defining the high-fluence contour by outlining a perimeter of the group of pixels. The percentage of the highest intensity value is 70% or more, e.g., 80% or more. Alternatively, or additionally, defining a high-fluence contour may include calculating a center of mass of the radiation intensity map using intensity values of each pixel, and centering the treatment planning contour over the calculated center of mass. In some variations, determining the contour difference between the high-fluence contour and the treatment planning contour may include calculating a distance between the high-fluence contour and the treatment planning contour. Calculating the distance may include calculating distances between the high-fluence contour and the treatment planning contour in 2D and averaging the calculated distances. Optionally, calculating the distance may include calculating distances between the high-fluence contour and the treatment planning contour in 3D and averaging the calculated distances. In some variations, determining the contour difference between the high-fluence contour and the treatment planning contour may include determining an overlap region between a high-fluence region enclosed by the high-fluence contour and the treatment planning region enclosed by the treatment planning contour, and calculating a percentage overlap based on an area or volume of the overlap region and an area or volume enclosed by the treatment planning contour. The graphical representation may further include an anatomical image and the high-fluence contour and the treatment planning contour are overlaid on the anatomical image. Optionally, the anatomical image may include one or more of a CT image and an MRI image. In some variations, the treatment planning contour may be a contour on a planning fluence map.

Also described herein are methods for determining a delivered radiation dose. One variation of a method may include emitting radiation beamlets from a radiation source of a radiotherapy system, wherein the emitted radiation beamlets are defined by openings of a multi-leaf collimator (MLC) located in front of the radiation source, and generating a delivered radiation dose map using measurements acquired by sensors of the radiotherapy system and CT image data. In some variations, the radiation beamlets may be emitted according to radiotherapy system machine instructions generated during a radiation delivery session. The sensors of the radiotherapy system may include a radiation imager, the acquired measurements comprise radiation imager data, and generating the delivered radiation dose map may include back projecting radiation imager data using inverse attenuation and the CT image data from a plurality of firing positions of a therapeutic radiation source and patient platform positions. Generating the delivered radiation dose map may further include mapping the radiation imager data to the CT image data on a row-by-row basis, deriving an attenuation coefficient for each pixel of the CT image data, calculating an amount of energy deposited to a pixel of the CT image data using the attenuation coefficient, generating an energy deposition map by combining the energy deposited to each pixel of the CT image data from all radiation beamlets emitted by the therapeutic radiation and firing positions, and calculating the delivered radiation dose from the energy deposition map. In some variations, the acquired radiation imager measurements may include radiation imager data acquired from multiple radiation source locations. Calculating the amount of energy deposited to each pixel of a CT image data may include calculating an unattenuated radiation imager signal for each pixel by reversing an attenuation of the radiation imager data according to the attenuation coefficient, and calculating the amount of energy deposited to each pixel of the CT image data based on the unattenuated radiation imager signal. In some variations, reversing the attenuation of the radiation imager data comprises increasing an intensity of the radiation imager data by an increment determined by the inverse-square law, and adjusting the increased intensity by the attenuation coefficient:

$$I ( x , y ) = I ( x , y ) \cdot {e}^{CT ( x , y ) \cdot Voxelsize / cos ( x , y )} .$$

The sensors of the radiotherapy system may include MLC leaf position sensors and a dose chamber disposed in a beam path of the radiation source. In some variations, generating the delivered radiation dose map may include forward projecting the MLC openings using MLC leaf position sensor data, dose chamber data, and the CT image data to calculate radiation deposition along the emitted radiation beamlets. Emitting radiation beamlets may include emitting radiation beamlets over a plurality of firing positions of the radiation source, and generating the delivered radiation dose map may include generating a delivered dose for the radiation beamlets emitted at each of the plurality of firing positions and summing the delivered doses over the plurality of firing positions. The radiotherapy system may further include a patient couch movable to a plurality of beam station positions, and emitting radiation beamlets may further include emitting radiation beamlets over the plurality of beam station positions. Generating the delivered radiation dose map may include generating a beam station delivered dose for the radiation beamlets emitted at each of the plurality of patient couch positions and summing the beam station delivered dose over the plurality of beam station positions. In some variations, the CT image data may be acquired on the same day as emitting the radiation beamlets. Optionally, some methods may include generating a graphical representation that includes the delivered radiation dose map. The graphical representation may further include an anatomical image and the delivered radiation dose map is overlaid with the anatomical image. In some variations, the graphical representation may further include a treatment planning contour and the delivered radiation dose map may be overlaid with the treatment planning contour.

Optionally, some methods may further include further calculating a gamma [symbol] metric value for a pre-determined distance-to-agreement criterion (C_{DTA}) and a predetermined percent dose different criterion (C_{DD}) and determining with the calculated gamma metric value meets a pre-determined threshold, wherein calculating the gamma [symbol] metric value comprises calculating, for each pixel on the delivered radiation dose map, a distance-to-agreement value (DTA) to a planned radiation dose map and a percent dose difference (DD) to the planned radiation dose, where the gamma metric value is given by:

$$γ = \sqrt{{( \frac{DTA}{{C}_{DTA}} )}^{2} + {( \frac{DD}{{C}_{DD}} )}^{2}} .$$

Alternatively, or additionally, methods may include calculating a delivered dose volume histogram (DVH) based on the radiation dose map. Methods may include comparing the delivered DVH with a planned DVH, and generating a graphical representation that includes the delivered DVH overlaid with the planned DVH. In some variations, the planned DVH may be a bounded DVH having a minimum DVH curve and a maximum DVH curve, and comparing the delivered DVH with the planned DVH comprises determining a number of points on the delivered DVH that are within bounds defined the minimum DVH curve and the maximum DVH curve. The graphical representation may include visual indicia indicating whether the number of points on the delivered DVH meet or exceed a predetermined threshold. Optionally, some methods may further include calculating a percentage of points on the delivered DVH that are within bounds defined the minimum DVH curve and the maximum DVH curve and the graphical representation may include visual indicia indicating whether the percentage of points meet or exceed a predetermined threshold.

Described herein are methods for evaluating the quality of a fluence map and radiotherapy system. One variation of a method may comprise delivering radiation according to a first radiation fluence map using a radiotherapy system comprising a linear accelerator (linac) and a MV detector located across from the linac, recording imaging data from the MV detector during radiation delivery, generating a second radiation fluence map using the recorded imaging data, determining a fluence map difference by comparing the first radiation fluence map and the second radiation fluence map to determine a difference, and if the fluence map difference is not within an acceptable range, generating a notification comprising an indication that the first radiation fluence map and the radiotherapy system are not of acceptable quality. In some variations, the first radiation fluence map may be a planned radiation fluence map intended for delivery to a target region, or the first radiation fluence map may be a quality assurance (QA) radiation fluence map that is not intended for delivery to a target region.

### BRIEF DESCRIPTION OF THE DRAWINGS

FIG. 1A is a block diagram of one variation of radiotherapy system.

FIG. 1B is a schematic perspective view of one variation of a radiotherapy system.

FIG. 1C is a perspective component view of one variation of a radiotherapy system.

FIG. 1D is another perspective view of one variation of a radiotherapy system.

FIG. 1E is a schematic depiction of one variation of a radiotherapy system.

FIG. 1F is a schematic depiction of one variation of a radiotherapy system.

FIG. 2A is a flowchart representation of one variation of a method of verifying radiotherapy system function.

FIG. 2B is a flowchart representation of one variation of a method for determining an MLC leaf location or position.

FIG. 2C is a flowchart representation of one variation of a method for determining an MLC leaf dwell time.

FIG. 3 is a flowchart representation of one variation of a method for evaluating linac function.

FIG. 4A is a schematic representation of a radiation beamlet path through a cross-sectional depiction of a patient.

FIG. 4B is a flowchart representation of one variation of a method for verifying a radiation beamlet path.

FIG. 4C is a flowchart representation of one variation of a method for determining a radiation beamlet path.

FIG. 4D is a flowchart representation of one variation of a method for determining a radiation beamlet path.

FIG. 5 is a flowchart representation of one variation of a method for comparing the delivered fluence with a location of a treatment contour.

FIG. 6 is a flowchart representation of one variation of a method for determining a cumulative delivered radiation dose.

FIG. 7A is a flowchart representation of one variation of a method for determining a cumulative delivered radiation dose.

FIG. 7B is an image of simulated radiation dose distribution generated by a treatment planning system. FIG. 7C is a plot of the dose values along line 7C-7C of the image of FIG. 7B.

FIG. 7D is an image of a delivered radiation dose distribution reconstructed from measured image data by radiation imagers of a radiotherapy system. FIG. 7E is a plot of the dose values along line 7E-7E of the image of FIG. 7D.

FIG. 8A is a flowchart representation of one variation of a method for monitoring radiation delivery.

FIG. 8B is a flowchart representation of one variation of a method for monitoring radiation delivery.

FIG. 9 is a flowchart representation of one variation of a method for evaluating the quality of a radiation fluence map and the performance of a radiotherapy system.

### DETAILED DESCRIPTION

Disclosed herein are radiotherapy systems (also referred to as radiotherapy delivery systems) and methods for evaluating whether the radiation emitted by a therapeutic radiation source delivers a desired amount of radiation dose to the planned target region(s) in accordance with a treatment plan. Radiotherapy systems may comprise one or more radiation detectors (also referred to as radiation imagers) that are configured to measure the radiation emitted by a therapeutic radiation source. For example, a radiotherapy system may comprise a radiation imager that is located across from (e.g., opposite) the therapeutic radiation source. The methods described herein may comprise using measurements from the radiation imager, and optionally in conjunction with information from treatment planning, may be used to determine the amount of radiation emitted (e.g., radiation fluence), what region in the patient (or phantom) was irradiated, and whether the amount and spatial distribution of the radiation fluence is within an acceptable range of the planned radiation fluence. In some variations, a radiation dose to the patient (or phantom) may be calculated by combining the radiation fluence information with CT imaging data and/or attenuation measurements. The CT imaging data may be acquired, in some variations, at the beginning of the treatment session. These evaluations may be performed during the treatment session (e.g., in real-time) and/or may be performed at the conclusion of the treatment session. In some variations, the methods described herein may be used during a quality assurance (QA) session, in the absence of a patient. Measuring, monitoring, and verifying the emitted radiation during a radiation delivery session, such as a treatment session or a QA session, especially in real-time, may help ensure that the radiotherapy treatment is proceeding according to a clinician-approved treatment plan and may help promote patient safety by generating a notification if the radiation delivery or radiotherapy treatment deviates from the treatment plan.

Additionally, or alternatively, the methods described herein may be used to verify that the radiotherapy system components are working as expected. For example, radiation imager data may be combined with MLC actuation instructions to verify that the MLC leaves were opened or closed (and/or otherwise moved or positioned) according to the MLC actuation instructions from the radiotherapy delivery system controller. Alternatively, or additionally, radiation imager data may be combined with therapeutic radiation source instructions (e.g., pulse sequence, timing, and/or energy) to verify that the therapeutic radiation source emitted radiation according to the instructions from the radiotherapy delivery system controller. Monitoring the functionality of these radiotherapy system components during the delivery of radiation (whether it be during a treatment session or a quality assurance or calibration session) may be a primary or confirmatory check that the radiation delivery is proceeding as expected. In some variations, the methods described herein may be used in conjunction with MLC position sensors (e.g., Hall sensors coupled to each leaf of an MLC) and/or a dose chamber associated with (e.g., located within the generated radiation field of) the therapeutic radiation source. In this way, there may be secondary confirmation that the radiation delivery is proceeding as intended and/or the delivery systems components are functioning properly. In some variations, these methods may be used during a QA session, where radiation is delivered in the absence of a patient.

### Radiotherapy Systems

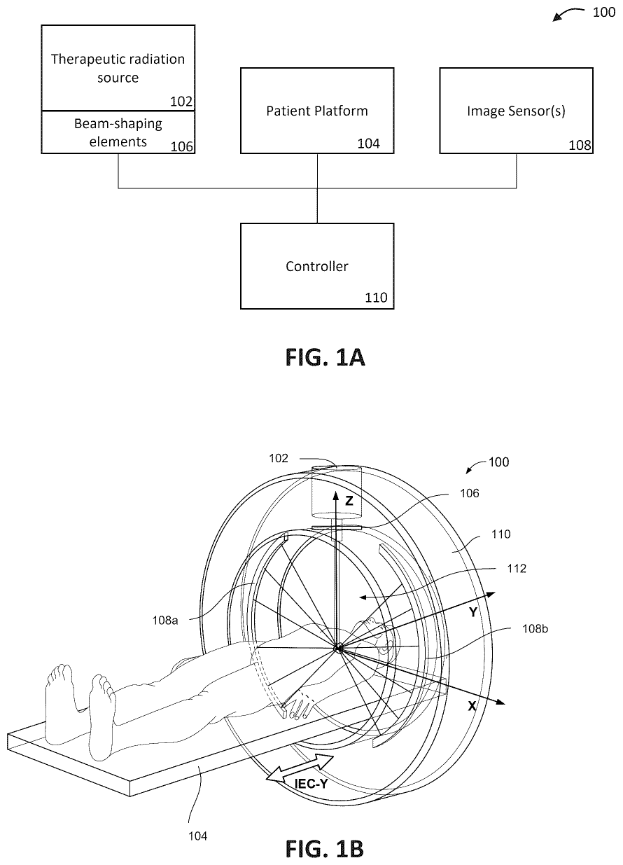

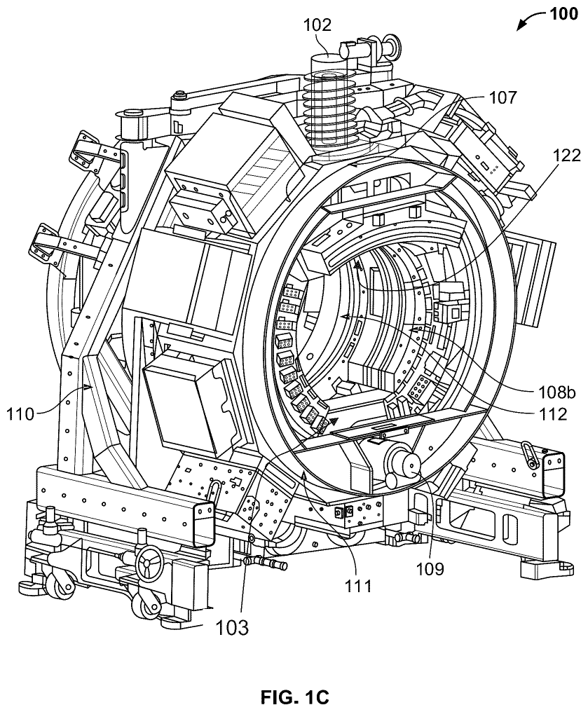

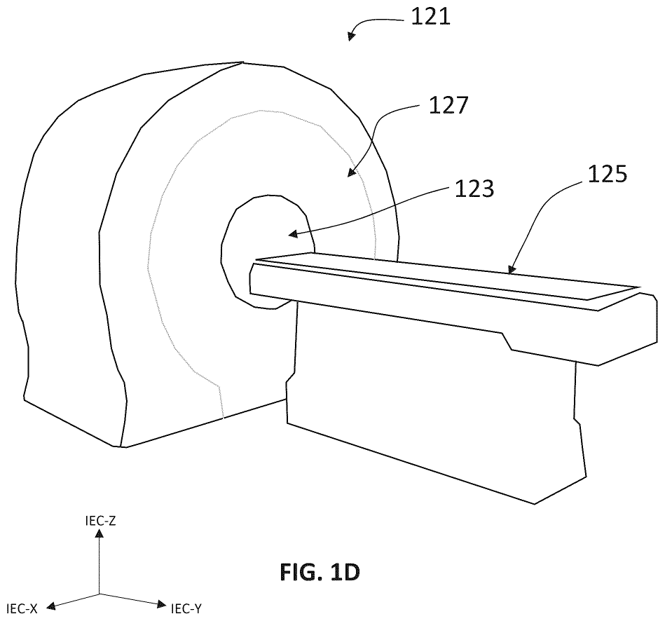

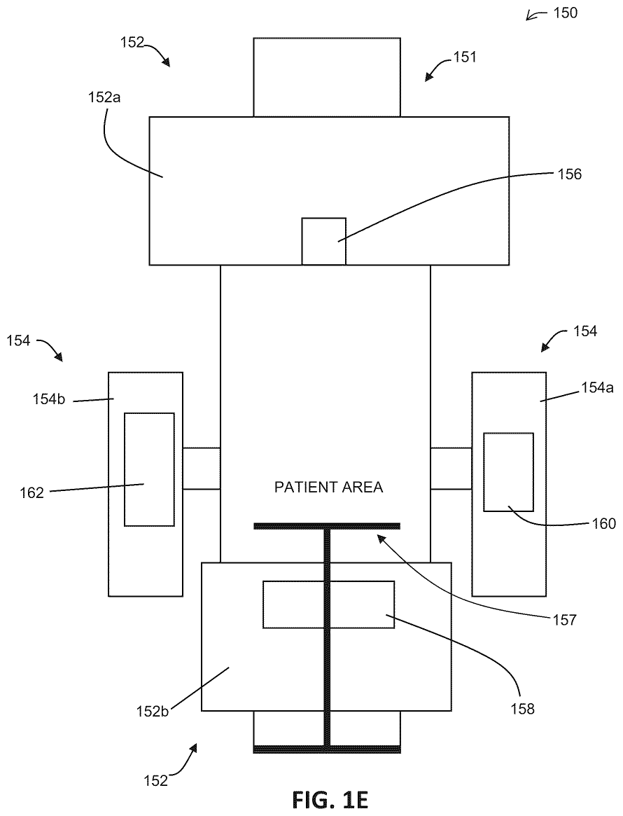

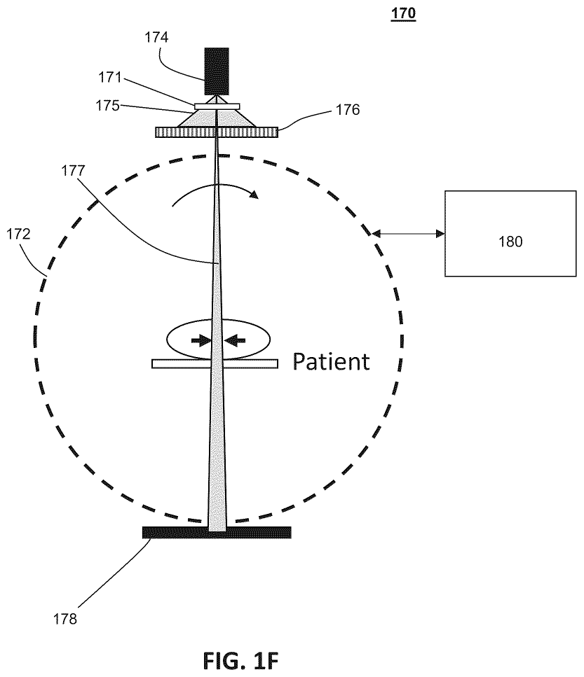

The methods described herein may be used with various radiotherapy systems, for example, any radiotherapy system comprising a therapeutic radiation source configured to emit high-energy photons and a radiation detector (also referred to as a radiation imager) located opposite the therapeutic radiation source. FIG. 1A depicts a functional block diagram of a variation of a radiotherapy system that may be used with one or more of the methods described herein. Radiotherapy system (100) comprises one or more therapeutic radiation sources (102) and a patient platform (104). The therapeutic radiation source may comprise an X-ray source, electron source, proton source, a neutron source, and/or any suitable particles including carbon atoms. For example, a therapeutic radiation source (102) may comprise a linear accelerator (linac), Cobalt-60 source, and/or an X-ray machine. The therapeutic radiation source may be movable about the patient platform so that radiation beams may be directed to a patient on the patient platform from multiple firing positions and/or angles. In some variations, a radiotherapy system may comprise one or more beam-shaping elements and/or assemblies (106) that may be located in the beam path of the therapeutic radiation source. For example, a radiotherapy system may comprise a linac (102) and a beam-shaping assembly (106) disposed in a path of the linac radiation beam. The beam-shaping assembly may comprise one or more movable jaws and one or more collimators. At least one of the collimators may be a multi-leaf collimator (e.g., a binary multi-leaf collimator, a 2-D multi-leaf collimator, etc.), which may comprise a plurality of independently movable leaves. The linac and the beam-shaping assembly may be mounted on a gantry or movable support frame that comprises a motion system configured to adjust the position of the linac and the beam-shaping assembly. In some variations, the linac and beam-shaping assembly may be mounted on a support structure comprising one or more robotic arms, C-arms, gimbals, and the like. The patient platform (104) may also be movable. For example, the patient platform (104) may be configured to translate a patient linearly along a single axis of motion (e.g., along the IEC-Y axis), and/or may be configured to move the patient along multiple axes of motion (e.g., 2 or more degrees of freedom, 3 or more degrees of freedom, 4 or more degrees of freedom, 5 or more degrees of freedom, etc.). In some variations, a radiotherapy system may have a 5-DOF patient platform or a 6-DOF patient platform that is configured to move along the IEC-Y axis, the IEC-X axis, the IEC-Z axis, as well as pitch, yaw, and/or roll.

In the variation shown in FIG. 1A, the radiotherapy system (100) also comprises a controller (110) that is in communication with the therapeutic radiation source (102), beam-shaping elements or assemblies (106), patient platform (104), and one or more image sensors (108) (e.g., one or more imaging systems). The controller (110) may comprise one or more processors and one or more machine-readable memories in communication with the one or more processors, which may be configured to execute or perform any of the methods described herein. The one or more machine-readable memories may store instructions to cause the processor to execute modules, processes and/or functions associated with the system, such as one or more treatment plans, the calculation of radiation fluence maps based on treatment plan and/or clinical goals, segmentation of fluence maps into radiotherapy system instructions (e.g., that may direct the operation of the gantry, therapeutic radiation source, beam-shaping assembly, patient platform, and/or any other components of a radiotherapy system), and image and/or data processing associated with treatment planning and/or radiation delivery. In some variations, the memory may store treatment plan data (e.g., treatment plan firing filters which may be transform functions that convert imaging data into fluence maps, fluence maps, beamlet sequence, planning images, treatment session PET pre-scan images and/or initial CT, MRI, and/or X-ray images), and instructions for delivering the derived fluence map (e.g., instructions for operating the therapeutic radiation source, beam-shaping assembly and patient platform in concert). In some variations, the controller (110) may comprise a processor for each of the therapeutic radiation source, beam-shaping elements, patient platform, and/or image sensors, with corresponding machine-readable memories. For example, in one variation, there may be an MLC processor and corresponding machine readable memory that generates and/or stores MLC instructions that specify the position, motion, location, timing (e.g., transition or travel time, dwell time, etc.) of each of the leaves of the MLC. The MLC instructions may be generated by a treatment planning system and stored in the memory associated with the MLC processor. Alternatively, or additionally, the MLC instructions may be generated by the MLC processor (e.g., in real-time) by the MLC processor, based on updated fluence maps and/or fluence maps generated based on imaging data (e.g., biology-guided radiotherapy which generates fluence maps from newly-acquired biologically-related imaging data, such as PET data). The controller of a radiotherapy system may be connected to other systems by wired or wireless communication channels. For example, the radiotherapy system controller may be in wired or wireless communication with a radiotherapy treatment planning system controller such that fluence maps, firing filters, initial and/or planning images (e.g., CT images including cone beam CT images and fan beam CT images, MRI images, PET images, 4-D CT images), imaging data, patient data, and other clinically-relevant information may be transferred from the radiotherapy treatment planning system to the radiotherapy system. The delivered radiation fluence, any dose calculations, and any clinically-relevant information and/or data acquired during the treatment session may be transferred from the radiotherapy system to the radiotherapy treatment planning system. This information may be used by the radiotherapy treatment planning system for adapting the treatment plan and/or adjusting delivery of radiation for a successive treatment session.

FIG. 1B depicts one variation of a radiotherapy system (100). Radiotherapy system (100) may include a gantry (110) rotatable about a patient treatment region (112), one or more PET detectors (108) mounted on the gantry, a therapeutic radiation source (102) mounted on the gantry, a beam-shaping module (106) disposed in the beam path of the therapeutic radiation source, and a patient platform (104) movable within the patient treatment region (112). In some variations, the gantry (110) may be a continuously-rotatable gantry (e.g., able to rotate through 360° and/or in arcs with an angular spread of less than about 360°). The gantry (110) may be configured to rotate from about 20 RPM to about 70 RPM about the patient treatment region (112). For example, the gantry (110) may be configured to rotate at about 60 RPM. The gantry may also be configured to rotate at a slower rate, e.g., 20 RPM or less, 10 RPM or less, 1 RPM or less. The beam-shaping module (106) may include a movable jaw and a dynamic multi-leaf collimator (MLC) having one or more independently movable leaves. The beam-shaping module may be arranged to provide variable collimation width in the longitudinal direction of 1 cm, 2 cm, or 3 cm at the system iso-center (e.g., the rotation center of the system and on the central treatment or imaging plane, the center of a patient treatment area). The jaw may be located between the therapeutic radiation source and the MLC or may be located below the MLC. Alternatively, the beam-shaping module may include a split jaw where a first portion of the jaw is located between the therapeutic radiation source and the MLC, and a second portion of the jaw is located below the MLC and coupled to the first portion of the jaw such that both portions move together. The therapeutic radiation source (102) may be configured to emit radiation at predetermined firing positions (e.g., firing angles 0°/360° to 359°) about the patient treatment region (112). For example, in a system with a continuously-rotatable gantry, there may be from about 50 to about 100 firing positions (e.g., 50 firing positions, 60 firing positions, 80 firing positions, 90 firing positions, 100 firing positions, etc.) at various angular positions (e.g., firing angles) along a circle circumscribed by the therapeutic radiation source as it rotates. The firing positions may be evenly distributed such that the angular displacement between each firing position is the same.

FIG. 1C depicts a perspective component view of the radiotherapy system (100). As shown there, the beam-shaping module may further include a primary collimator or jaw (107) disposed above a binary MLC (122) (partially obscured). A binary MLC is a multi-leaf collimator where each leaf may be individually and independently retained at, and moved between, an open position and a closed position. The radiotherapy system may also include an MV X-ray detector (103) located opposite the therapeutic radiation source (102). Optionally, the imaging system of the radiotherapy system (100) may further include a kV CT imaging system on a ring (111) that is attached to the rotatable gantry (110) such that rotating the gantry (110) also rotates the ring (111). The kV CT imaging system may include a kV X-ray source (109) and an X-ray detector (115) located across from the X-ray source (109). The therapeutic radiation source or linac (102) and the PET detectors (108a) (not depicted; located opposite 108b) and (108b) may be mounted on the same cross-sectional plane of the gantry (i.e., PET detectors are co-planar with a treatment plane defined by the linac and the beam-shaping module), while the kV CT scanner and ring may be mounted on a different cross-sectional plane (i.e., not co-planar with the treatment plane). The radiotherapy system (100) of FIGS. 1B and 1C may have a first imaging system that includes the kV CT imaging system and a second imaging system that includes the PET detectors. Optionally, a third imaging system may include the MV X-ray source (e.g., therapeutic radiation source, linac) and a radiation imager (e.g., MV detector). The imaging data acquired by one or more of these imaging systems may include X-ray and/or PET imaging data, and the radiotherapy system controller may be configured to store the acquired imaging data and calculate a radiation delivery fluence using the imaging data, for example, in a biology-guided radiotherapy (BgRT) session. Some variations may further include patient sensors, such as position sensors and the controller may be configured to receive location and/or motion data from the position sensor and incorporate this data with the imaging data to calculate a radiation delivery fluence. Additional descriptions of radiotherapy systems that may be used with any of the methods described herein are provided in U.S. Pat. No. 10,695,586, filed Nov. 15, 2017, which is hereby incorporated by reference in its entirety.

FIG. 1D depicts an example of a radiotherapy system (121) that comprises a circular or ring gantry (enclosed in system housing or covers (127)) that has a bore (123), and a patient platform (125) movable through the bore. The radiotherapy system may comprise a rotatable gantry and a therapeutic radiation source and a radiation detector mounted on the rotatable gantry. In one variation, the therapeutic radiation source may be a high-energy photon source such as a linear accelerator (linac) and the radiation detector may be an MV detector mounted opposite the linac. Optionally, the radiotherapy system may comprise one or more imaging systems, such as a kV CT imaging system and an optional PET imaging system, which may be mounted on the gantry or mounted on a structure adjacent to the rotatable gantry. In some variations, the kV CT imaging system is in-plane with the linac (i.e., the imaging plane overlaps with the radiation beamlets that comprise the treatment plane).

The patient platform (104, 125) may be movable in the treatment region (112) to discrete, pre-determined locations along IEC-Y. These discrete, pre-determined locations may be referred to as “beam stations”. In one variation, different beam stations may vary only by their location along the IEC-Y axis (e.g., longitudinal axis); each beam station may be identified by its location along IEC-Y. Alternatively, or additionally, beam stations may vary by the platform pitch, yaw, and/or roll positions of the patient platform. For example, a radiotherapy treatment planning system may specify 200 beam stations, where each beam station is about 2 mm (e.g., 2.1 mm) apart along IEC-Y from its adjacent beam stations. During a treatment session, the radiotherapy treatment system may move the patient platform to each of the beam stations and may stop the platform at a beam station while radiation is delivered to the patient. In some variations, after the platform has been stepped to each of the 200 beam stations in a first direction (e.g., into the bore), the platform may be stepped to each of the 200 beam stations in a second direction opposite the first direction (e.g., out of the bore, in reverse), where radiation is delivered to the patient while the platform is stopped at a beam station. When the platform has been stepped to a beam station, the platform is stopped while radiation is delivered. Alternatively, or additionally, after the platform has been stepped to each of the 200 beam stations in a first direction (e.g., into the bore) where radiation is delivered at each of the beam stations, the platform may be moved in reverse so that it returns to the first beam station. No radiation may be delivered while the platform is moved back to the first beam station. The platform may then be stepped, for a second time, to each of the 200 beam stations in the first direction for a second pass of radiation delivery. In some variations, the platform may be moved continuously while radiation is delivered to the patient and may not be stopped at beam stations during the delivery of therapeutic radiation. Additional descriptions of patient platforms that may be used with any of the radiotherapy systems and methods described herein are provided in U.S. Pat. No. 10.702.715, filed Nov. 15, 2017, which is hereby incorporated by reference in its entirety.

FIG. 1E depicts another variation of a radiotherapy system (150) that may be used to deliver radiation in accordance with any of the methods described herein. The radiotherapy system (150) may have the components of the radiotherapy system represented in the block diagram of FIG. 1. Radiotherapy system (150) may comprise a gantry or support structure (151) comprising a first pair of arms (152) rotatable about a patient area and a second pair of arms (154) rotatable about the patient area, an imaging system comprising a therapeutic radiation system comprising a high-energy photon source (156) (also referred to as an MV radiation source) mounted on a first arm (152a) of the first pair of arms (152) and an MV detector or radiation imager (158) mounted on a second arm (152b) of the first pair of arms (152), and a kV radiation source (160) mounted on a first arm (154a) of the second pair of arms (154) and a kV detector (162) mounted on a second arm (154b) of the second pair of arms (154). System (150) may also comprise beam-shaping elements or assemblies similar to those described above, e.g., comprising one or more jaws and multi-leaf collimator. The first and second arms of the first pair of arms (152) may be located opposite each other (e.g., on opposite sides of the patient area, across from each other, and/or about 180 degrees from each other), such that the MV radiation source (156) and the radiation imager (158) are located opposite each other (e.g., the radiation imager is located in the beam path of the MV radiation source). The first and second arms of the second pair of arms (154) may be located opposite each other (e.g., on opposite sides of the patient area, across from each other, and/or about 180 degrees from each other), such that the kV radiation source (160) and the kV detector (162) are located opposite each other (e.g., the kV detector is located in the beam path of the kV radiation source). The radiotherapy system controller may be configured to store acquired imaging data (from either or both the kV detector and the radiation imager) and calculate a radiation delivery fluence. Optionally, one or more target region surrogate devices, such as a breathing sensor, may be included, and the controller may be configured to receive location and/or motion data from the target region surrogate to calculate a radiation delivery fluence.

The MV radiation source (156) (i.e., the therapeutic radiation source) may be configured to emit radiation at predetermined firing positions about the patient area. The system may also comprise a patient platform or couch (157) that is movable within the patient area. In some variations where the MV radiation source is moved around the patient area along a single plane, the firing positions may be referred to as firing angles, which may be from 0°/360° to 359°. Alternatively, or additionally, the gantry and/or support structure arms may be configured to move the MV radiation source to a firing position at any coordinate(s) in 3-D space, i.e., as designated by coordinates (x,y,z). For example, the gantry arms (152, 154) may be robotic arms having articulated joints and/or one or more gimbals that may be configured to position and/or orient the MV radiation source at any desired firing position. The gantry or support structure may be configured to continuously move MV radiation source through the firing positions or may be configured to step the MV radiation source to each firing position (i.e., move the MV radiation source to a firing position and remain stationary at that firing position). Alternatively, or additionally, the MV radiation source may be configured to emit radiation only at the predetermined firing positions or may be configured to emit radiation continuously, even as it is being moved from one firing position to the next.

The radiation detector (also referred to as a radiation imager) located opposite the therapeutic radiation source may comprise an imager (e.g., any electronic portal imaging device EPID) may have an acquisition rate that is similar to, or faster than, the rate at which radiation beamlets are emitted from the therapeutic radiation source. Radiation beamlets may be defined by openings of a multi-leaf collimator (MLC) located in front of the therapeutic radiation source. In some variations, the radiation imager acquisition rate may be similar to, or faster than, the rate at which the MLC is able to transition its leaves. In other variations, the radiation imager acquisition rate may be similar to, or faster than, the rate at which the therapeutic radiation source changes its firing position. A radiation detector such as a radiation imager that has a rapid acquisition rate may facilitate the detection of radiation emitted by the therapeutic radiation source in real-time, with little or no latency between radiation beamlet emission by the linac and radiation detection by the radiation imager, which may support real-time monitoring of radiotherapy system operation. In some variations, a radiation imager having a particular acquisition rate may be selected based at least in part on the rotation speed of the gantry and/or the actuation speed of the MLC leaves. The radiation imager may acquire radiation measurements at a rate that is comparable to the gantry rotation speed and/or MLC leaves. That is, the faster the gantry rotation speed and/or MLC leaf actuation, the faster the radiation imager acquisition rate needs to be in order to capture the changes in emitted radiation. For example, the acquisition rate of a radiation imager that may be used with any of the methods described herein may be from about 15 frames/second to about 150 frames/second, e.g., from about 50 frames/second to about 70 frames/second, from about 70 frames/second to about 150 frames/second, about 50 frames/second, about 100 frames/second. In some variations, a radiotherapy system may comprise a radiation imager having an acquisition rate of about 100 frames/second or more. Some variations may comprise a strip radiation imager. A radiation imager with a strip geometry may be a detector where the radiation sensing surface has a width that is substantially smaller (e.g., narrower) than its length. For example, the length-to-width ratio of a strip radiation imager may be from about 5:1 to about 50:1, e.g., about 10:1, 20:1, 25:1, 30:1, etc. In one variation, a strip radiation imager may have a width along a Y-axis (e.g., IEC-Y axis) of about 2 cm and a length along an X-axis (e.g., IEC-X axis) of about 80 cm. Alternatively, or additionally, the width of a radiation imager may be determined at least in part on a dimension of the radiation field of the therapeutic radiation source. For example, the width of the radiation imager may be less than the width of the therapeutic radiation field.

Some variations of a radiotherapy system for use with the methods and systems described herein may comprise a binary multi-leaf collimator (bMLC) where each leaf may be independently controlled by the MLC processor to be in either an open position (where a radiation beamlet passes through the opening created by the leaf) or a closed position (where a radiation beam is blocked by the leaf). The bMLC may be configured to transition a leaf between the open and closed positions at a rate from about 100 Hz to about 150 Hz, e.g., about 100 Hz, about 125 Hz. FIG. IF depicts a schematic representation of one variation of a radiotherapy system (170) comprising a rotatable gantry (172) configured to rotate up to about 70 RPM (e.g., about 60 RPM), a therapeutic radiation source (e.g., linac 174) mounted on the gantry, a bMLC (176) disposed in the beam path of the therapeutic radiation source, and a strip radiation imager (178) mounted on the gantry directly opposite the linac. There may also be a dose chamber (171) disposed in the radiation field (175) of the linac (174) that is configured to measure the radiation fluence output by the linac. In some variations, the bMLC may be focused to the therapeutic radiation source. The linac (174) may emit a radiation field (175) that may be collimated into a radiation beam (177) by the bMLC (176). The strip radiation imager (178) may have an acquisition rate of at least 15 frames/second (e.g., 50 frames/second, 120 frames/second, 130 frames/second, 140 frames/second, 150 frames/second), and may be about 80 cm in length (along IEC-X) and about 3 cm in width (along IEC-Y). In some variations, the strip radiation imager may have a width of 2 cm (along IEC-Y) at system isocenter. The radiotherapy system (170) may also comprise a controller (180), which may include a plurality of subsystems including, but not limited to, a central processor, an MLC processor, and an image processor. One or more subsystems of the controller (180) contain programming instructions to perform one or more calculations and data collection and processing, and may also be in communication with the therapeutic radiation source, bMLC, gantry, radiation imager and/or the patient platform. The radiotherapy system may be configured to receive information from a treatment planning system, via a data connection, which may be wired connection, wireless connection, and/or internet cloud connection. Alternatively, or additionally, the data transfer may occur via a data transfer medium (e.g., portable memory drive, disks).

### Methods

One variation of a method for monitoring the operation of a radiotherapy system may use radiation measurements acquired by a radiation detector (such as an EPID that is configured to sense radiation having energy in the megavolt range, also referred to as an MV detector or radiation imager) to determine whether the beam-shaping assembly is functioning in accordance with the instructions from the system controller. For example, some methods may use radiation measurements from the radiation imager to determine whether MLC leaf motion characteristics (e.g., leaf speed, leaf dwell time) and/or MLC leaf position is in accordance with the MLC actuation instructions or commands from the MLC processor of the system controller. The MLC actuation instructions may be generated by the treatment planning system and transferred to the radiotherapy system, and/or may be generated by the radiotherapy system controller based on a planned fluence map. The MLC leaf motion and/or position calculated from the radiation imager data may be compared against the MLC pattern or MLC actuation instructions generated by the treatment planning system and/or the MLC pattern or MLC actuation instructions generated by the radiotherapy system controller. In some variations, the MLC pattern or MLC actuation instructions may be generated by the radiotherapy system controller during the radiation delivery session. Other methods may use radiation measurements from the radiation imager to determine whether the therapeutic radiation source (e.g., linac) is operating in accordance with the linac instructions or commands from the radiotherapy controller. The methods described herein may be used during a treatment session where radiation is emitted by the therapeutic radiation source to a patient on a patient platform, according to a treatment plan. The methods described herein may also be used during a calibration and/or quality assurance (QA) session where radiation is emitted by the therapeutic radiation source according to the treatment plan in the absence of a patient. Optionally, a phantom may be placed on the platform instead of a patient, for example, as part of a calibration and/or QA session. During a calibration and/or QA session, radiation measurements may be acquired by the radiation imager mounted on the radiotherapy system, radiation detectors placed on the platform, and/or radiation detectors in the optional phantom.

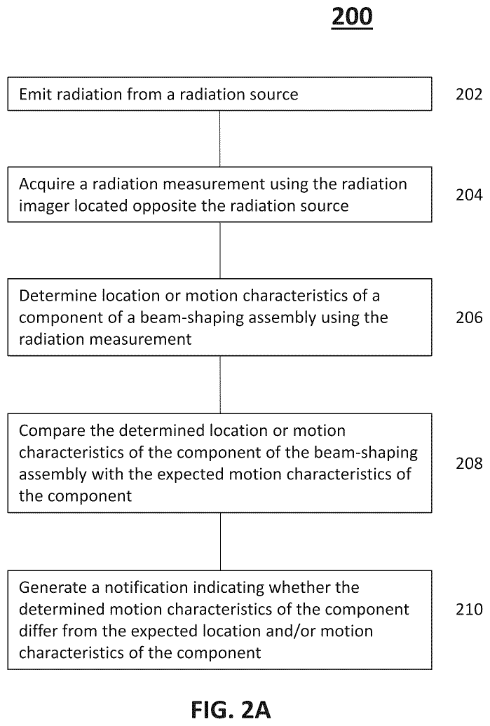

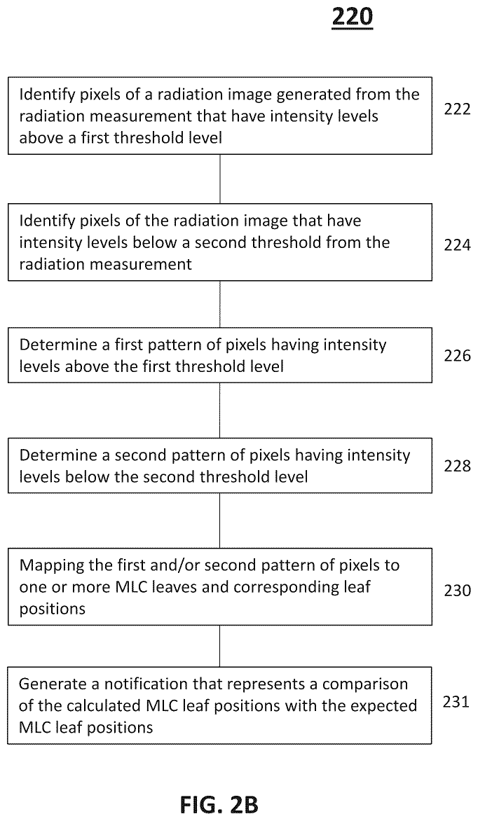

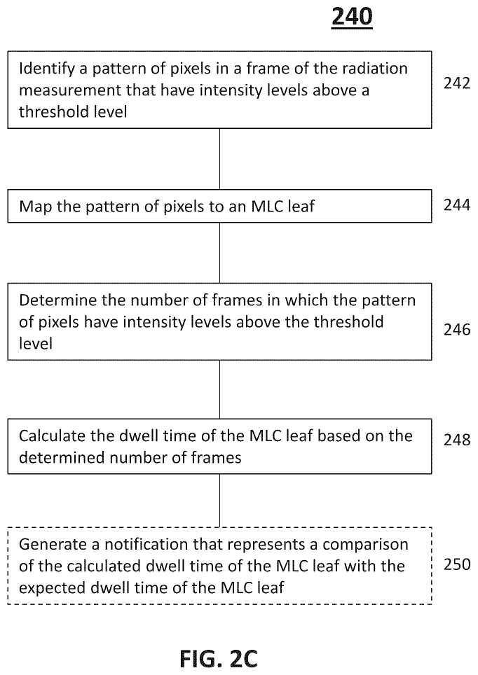

FIG. 2A depicts a flowchart representation of one variation of a method of verifying MLC function using radiation measurements acquired by a radiation imager that is located opposite the therapeutic radiation source. Method (200) may comprise emitting (202) radiation from a radiation source, acquiring (204) a radiation measurement using a radiation imager located opposite the radiation source, determining (206) motion characteristics of a component of a beam-shaping assembly using the radiation measurement, comparing (208) the determined motion characteristics with an expected motion characteristics, and generating (210) a notification indicating whether the determined location and/or motion characteristics of the component differ from the expected location and/or motion characteristics of the component. In some variations, the radiation source may be a therapeutic radiation source such as a linac, and the radiation imager may be an MV detector. Both the therapeutic radiation source, and the radiation imager may be mounted on a gantry or arm of the radiotherapy system. A beam-shaping assembly may comprise one or more jaws and/or MLCs with a plurality of independently actuatable leaves. While the method (200) may be used to may be used to verify the function of the MLC, it may also be used to verify the function of one or more jaws of the beam-shaping assembly. In one example, the beam-shaping assembly may comprise a binary MLC. Motion characteristics of the beam-shaping assembly may include, but are not limited to, the location of the jaw(s) and/or MLC leaves, the amount of time the jaw(s) and/or MLC leaves spend at a location (also referred to as dwell time), the speed at which the jaw(s) and/or MLC leaves move. In some variations where the beam-shaping assembly has a bMLC, motion characteristics may include whether a leaf is in the open position or the closed position, or in an intermediate position, e.g., when there is a malfunction. The expected motion characteristics of a beam-shaping assembly component may be determined by the treatment planning system and/or the radiotherapy system controller. For example, the expected motion characteristics of each leaf of an MLC may be determined by the MLC processor, in accordance with a planned fluence map. Some radiotherapy system controllers are configured to translate or segment a fluence map into MLC instructions in real-time during radiation delivery. The MLC instructions may include the position, motion, dwell time, etc. of each MLC leaf. The expected motion characteristics may be derived from these MLC instructions and used as a reference for comparing the motion characteristics determined from the radiation measurements. In some variations, the motion characteristics of one or more MLC leaves as determined by the radiation measurements from the radiation imager may be compared with the motion characteristics of one or more leaves as measured by MLC leaf sensors (which may be considered the “expected” motion characteristics of the MLC leaves). An MLC may have position sensor for each leaf to monitor the motion characteristics of the leaf. In these variations, the radiation measurements may optionally be used as a secondary (e.g., back-up) verification of MLC operation. If there is a discrepancy between the motion characteristics and/or MLC leaf pattern as determined by the radiation measurements and the motion characteristics and/or MLC leaf pattern as determined by the MLC leaf sensors, a notification may be generated. The radiotherapy system operator may have the option to investigate or resolve these discrepancies before continuing with radiation delivery. This may be used as an optional additional safety feature to ensure proper radiotherapy system operation, which may be particularly desirable during a treatment session.

The radiation measurements from the radiation imager or radiation detector may be combined to form an image having an array of pixels that spatially correspond with the detector elements of the radiation imager, and where the intensity of each pixel is proportional to the amount of radiation detected at the corresponding detector element. Regions of the radiation image with bright pixels (e.g., having an intensity above an upper threshold) may correspond to areas where the MLC leaf (or leaves) are in an open configuration. Regions of the radiation image with dim pixels (e.g., having an intensity below a low threshold) may correspond to areas where the MLC leaf (or leaves) are in a closed configuration. The image processor of the radiotherapy system controller may be configured to analyze the radiation image to identify regions of high-intensity or low-intensity pixels, and along with the known position of the MLC relative to the radiation imager, map those regions to particular MLC leaves being in the open position or the closed position. In some variations, the image processor may be able to determine, from the radiation image, the position of the MLC leaves along the motion trajectory. FIG. 2B depicts one variation of a method (220) for determining an MLC leaf location or position using measurements from a radiation imager of a radiotherapy system. This method may be used with method (200), which is described and depicted in FIG. 2A. For example, this method may be used as part of step (206) of method (200). Method (220) may comprise identifying (222) pixels of a radiation image having intensity levels above a first threshold level, determining (226) a first pattern of pixels having intensity levels above the first threshold level, and mapping (230) the first pattern of pixels to one or more MLC leaves and corresponding leaf positions. In some variations, alternatively or additionally to identifying pixels of the radiation image that have an intensity above a first threshold level, method (220) may comprise identifying (224) pixels of the radiation image that have intensity levels below a second threshold from the radiation measurement and may comprise determining (228) a second pattern of pixels having intensity levels below the second threshold level. Method (220) may also comprise mapping the second pattern of pixels to one or more MLC leaves and corresponding leaf positions. In some variations, the threshold(s) may be spatially variant. In some variations, the patient or phantom in the beam may be taken into consideration in obtaining the threshold(s). Optionally, method (220) may include generating (231) a notification that represents a comparison of the calculated MLC leaf position(s) with the expected MLC leaf position(s). The expected position(s) of MLC leaves (e.g., either in the open position or closed position) may be determined by the treatment planning system and stored in the radiotherapy system controller, and/or may be determined by the MLC processor and storied in the MLC processor memory. Alternatively, or additionally, the expected position(s) of MLC leaves may be determined from the MLC leaf position sensors, which monitor the motion characteristics (e.g., location, speed of motion, etc.) of each leaf. In some variations, a notification may be generated if the number of leaves that are not at their expected positions exceeds a threshold.

In some variations, the radiation image generated from the radiation measurements from the radiation detector imager may be used to determine the time duration of a leaf at a particular location, i.e., dwell time of a leaf. One method of determining the dwell time of an MLC leaf may include determining the number of image frames acquired by the radiation imager that indicate a leaf is in a particular location. The number of frames a particular set of pixels corresponding to an MLC leaf have an intensity above a threshold intensity level, in combination with the frame rate of the radiation imager, may be used to determine the length of time the MLC leaf was at a location, for example, an open position. Alternatively, or additionally, the dwell time of an MLC leaf in a closed position may be determined based on the number of frames a particular set of pixels corresponding to an MLC leaf have an intensity below a threshold intensity level and the frame rate of the radiation imager. The rate or speed of MLC leaf motion may be determined by identifying the high-intensity pixels (and/or low-intensity pixels) and monitoring changes in the location of the high-intensity pixels (and/or low-intensity pixels). FIG. 2C depicts one variation of a method for determining the dwell time of an MLC leaf at an open position using radiation measurements from the radiation imager. Method (240) may comprise identifying (242) a pattern of pixels in a frame of the radiation measurement that have intensity levels above a threshold level, mapping (244) the pattern of pixels to an MLC leaf, determining (246) the number of frames in which the pattern of pixels have intensity levels above the threshold level, and calculating (248) the dwell time of the MLC leaf based on the determined number of frames. For example, the dwell time may be calculated by multiplying the number of frames in which the pattern of high-intensity pixels is maintained with the acquisition frame rate (in frames/sec) of the radiation imager. Alternatively, or additionally, the method (240) may be used to determine the dwell time of an MLC leaf at a closed position by identifying a pattern of pixels in a frame of the radiation measurement that have intensity levels below a threshold level, mapping the pattern of pixels to an MLC leaf, determining the number of frames in which the pattern of pixels have intensity levels below the threshold level, and calculating the dwell time of the MLC leaf at the closed position based on the determined number of frames. Optionally, method (240) may include generating (250) a notification that represents a comparison of the calculated dwell time of the MLC leaf with the expected dwell time of the MLC leaf. The expected dwell time of MLC leaves at a particular position (e.g., either in the open position or closed position) may be determined by the treatment planning system and stored in the radiotherapy system controller, and/or may be determined by the MLC processor and storied in the MLC processor memory. Alternatively, or additionally, the expected dwell time of MLC leaves may be determined from the MLC leaf position sensors, which monitor the motion characteristics (e.g., location, speed of motion, etc.) of each leaf. In some variations, a notification may be generated if the difference between the calculated and expected dwell time exceeds a threshold.

Generating a notification in any of the methods described above may comprise generating an audio and/or visual alert that indicates whether the MLC is in the expected configuration. For example, a notification may be generated to represent the comparison between the actual, calculated motion characteristics or MLC configuration (i.e., pattern of MLC leaves in the open position or the closed position) and the expected motion characteristics or MLC configuration. In some variations, a notification may be generated if the MLC configuration and/or motion characteristics of one or more MLC leaves deviates from what is expected according to configuration and/or characteristics stored in the MLC processor memory. The notification may be generated by the radiotherapy system controller. The notification may be an audio alert and/or a visual alert. The visual alert may include, or be displayed on, a graphical user interface (GUI) that has text indicating that the MLC is not operating as expected, and optionally, a graphical depiction of which leaf or leaves do not have the expected motion characteristics. The GUI may be output to a display of the radiotherapy system, for example, a display that is viewable and accessible to the operator of the radiotherapy system. The radiotherapy system operator may determine, based on the notification, whether to pause the delivery of therapeutic radiation. Alternatively, or additionally, the system may pause radiation delivery if the deviation exceeds a preset level.

The operation of the therapeutic radiation source (e.g., linac) may be verified using radiation measurements acquired by a radiation imager (which may be an MV detector) that is located opposite the therapeutic radiation source. For example, the radiation measurements may be used to calculate the intensity of the radiation emitted by the linac, and the calculated intensity may be compared with the expected or intended intensity that is stored in the radiotherapy system controller memory. The radiation intensity detected by the radiation imager may be a number of photons per pixel (or detection element of the radiation imager), energy or energies of the photons (e.g., cumulative photon energy per pixel), number of pulses of photons, and/or any arbitrary measurement value (e.g., voltage level, current level, etc.) of the MV detector that is proportional to the amount of radiation incident on the radiation imager. In some variations, methods may determine, based on the radiation measurements, whether the linac emitted the number of radiation pulses and/or at the time(s) designated by the commands or instructions from the radiotherapy system controller. For example, some methods may determine whether the linac emitted pulses at the frequency, duration, duty cycle, energy, etc. specified by the machine instructions or commands of the controller. The linac instructions may be generated by the treatment planning system and transferred to the radiotherapy system controller, and/or the linac instructions may be generated by the radiotherapy system controller, for example based on a fluence map, machine instructions stored in the memory of a radiotherapy system controller, and/or a pre-loaded radiotherapy treatment plan. In some variations, the linac instructions may be generated by the radiotherapy system controller during the radiation delivery session. Real-time segmentation is a method by which radiotherapy machine instructions, including but not limited to MLC instructions and linac instructions, are generated during a radiation delivery session and are not generated before the radiation delivery session. Any of the methods described herein may be used during a radiation delivery session that includes real-time segmentation methods to generate machine instructions “on the fly”. These methods may help provide additional verification that the radiotherapy system performance is according to the machine instructions generated in real-time (e.g., based on a fluence map and/or a pre-loaded radiotherapy treatment plan and/or imaging data acquired on the day of radiation delivery).

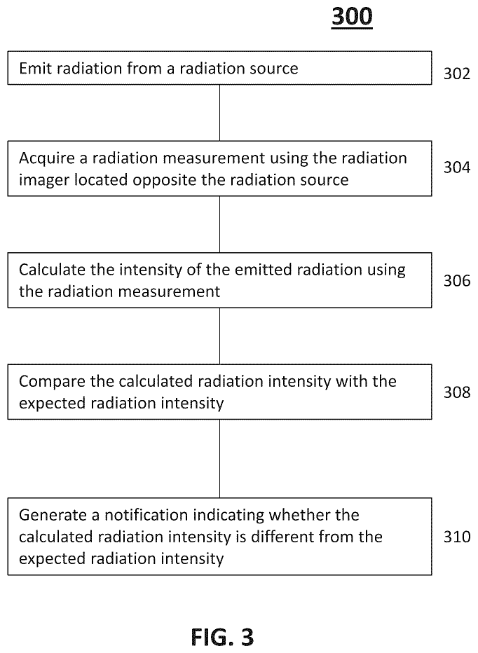

FIG. 3 depicts a flowchart representation of one variation of a method for verifying linac function using radiation measurements acquired by a radiation imager that is located opposite the linac. Method (300) may comprise emitting (302) radiation from the radiation source (e.g., therapeutic radiation source, linac), acquiring (304) radiation measurements using the radiation imager (e.g., radiation imager) located opposite the radiation source, calculating (306) the intensity of the emitted radiation using the radiation measurement, comparing (308) the calculated radiation intensity with the expected radiation intensity (308), and generating (310) a notification indicating whether the calculated radiation intensity is different from the expected radiation intensity. In some variations, acquired radiation measurements may be used to generate a radiation image where each pixel has an intensity value that represents the amount of radiation detected by the corresponding detector element of the radiation imager. The calculated intensity may be a number of photons detected per radiation image pixel and/or a cumulative photon energy detected by the detector element at a pixel over time. The changes of pixel intensity values over time may be used to determine the number of linac pulses that have been emitted, and the rate of pixel intensity changes may be used to determine the time characteristics (e.g., frequency, duration, duty cycle) of the linac pulses. The number and timing of linac pulses calculated based on the radiation measurements may be compared with the expected number and timing of linac pulses as determined by the radiotherapy system controller (either from the treatment planning system or generated by the radiotherapy system controller itself). In some variations, if the actual number and/or timing of linac pulses deviates from the expected number and/or timing of linac pulses, a notification (such as any of the notifications described above) may be generated. A GUI may display a visual notification, such as for example, text or graphics that depict the time at which the linac misfired or did not fire, and the expected linac firing pattern. The radiotherapy system may comprise a display device and the GUI may be output to the display device. The radiotherapy operator may then decide whether to pause radiotherapy based on the notification of linac operation.

### Verifying Radiation Beamlet Trajectory

The radiation measurements acquired by the radiation imager located opposite the therapeutic radiation source may be used to help determine whether the path an emitted radiation beamlet intersects the target region. This determination may be performed in real-time, and/or may be performed at set time intervals during radiation delivery. Acquiring multiple radiation measurements using the radiation imager as the linac rotates and delivers radiation may generate a pattern of radiation beamlet paths that intersect in space. The spatial extent of the intersection of radiation beamlet paths may be compared to the location of the target region to determine whether the radiotherapy system is emitting radiation to the target region. The radiation fluence delivered to a volume in space may be determined from the beamlet path data, which may provide an estimate of the radiation fluence delivered to the location of a target region. The radiation imager data, along with radiotherapy system component data (e.g., MLC leaf sensor data, linac sensor data, dose chamber data, gantry rotation data, patient platform motion data, etc.) may be used to calculate the path of a radiation beamlet. Optionally, the paths of the emitted radiation beamlets may be combined with attenuation data derived from a CT image to calculate the radiation dose to the target region or any volume in the treatment area.

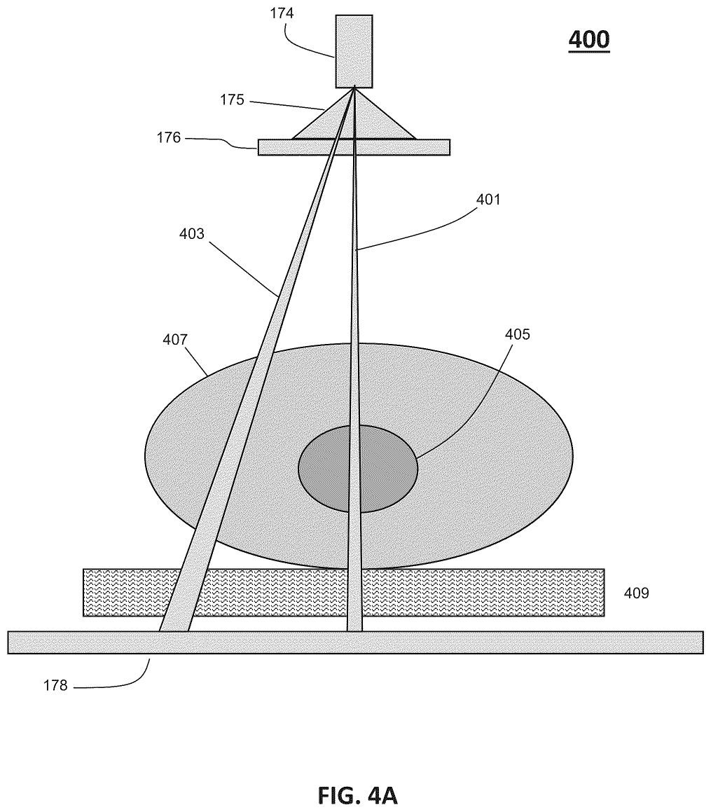

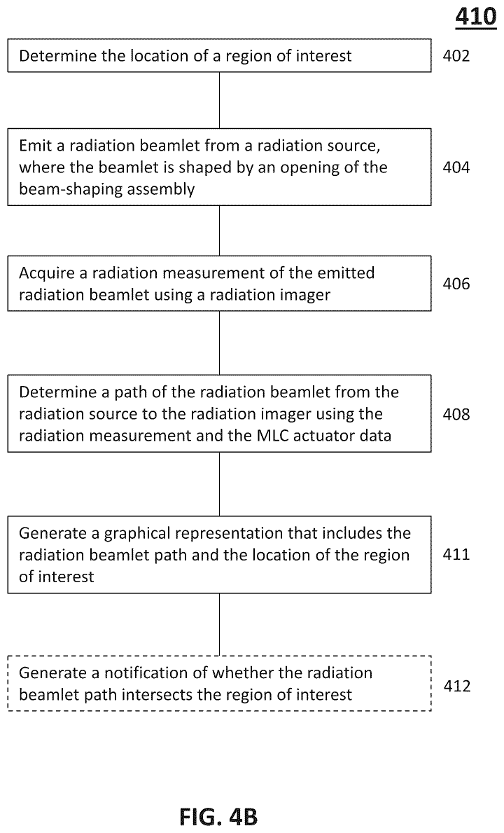

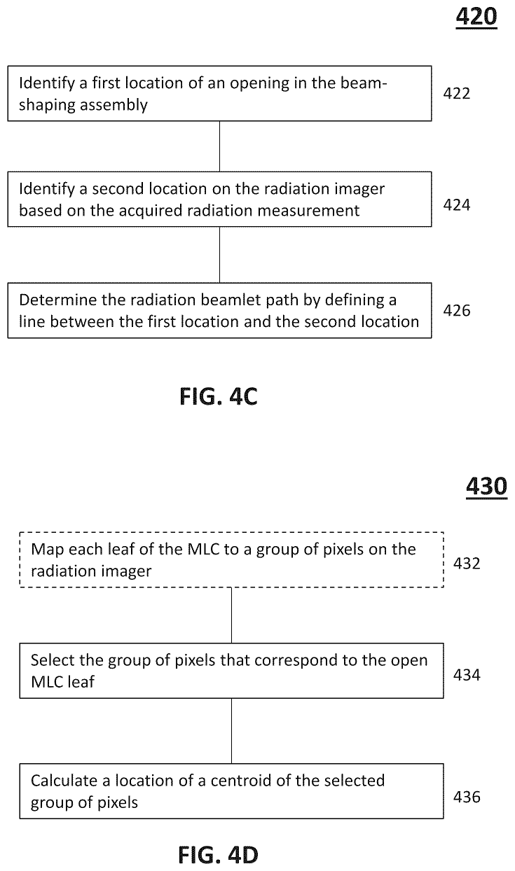

FIG. 4A schematically depicts the concept of radiation beamlet trajectory verification. The radiotherapy system (400) may comprise a linac (174), an MLC (176) located in a radiation field (175) emitted by the linac, and a radiation imager (e.g., MV detector) (178) located opposite the linac (174). The MLC (176) may shape or collimate the radiation field (175) into a radiation beamlet, such as radiation beamlet (401) or radiation beamlet (403). In some variations, the MLC (176) may be a bMLC. In this schematic, a patient (407) with a target region (405) is placed on the patient platform (409). Although this depicts a radiation delivery session with a patient (e.g., a treatment session), it should be understood that the methods described herein may be performed in the absence of a patient, for example, in a QA or calibration session where the patient is replaced with a phantom and/or an arrangement of radiation sensors to measure the radiation emitted by the linac (174). The trajectory of a particular radiation beamlet may be determined based on machine sensor data and radiation measurements acquired by the radiation imager (178). For example, an MLC may have position sensors for each of the leaves. The radiation beamlet trajectory for beamlet (401) may be calculated based on the location of the MLC leaf that is open and the location on the radiation imager (178) where a pulse of radiation was detected at the same time the linac (174) emitted a pulse of radiation. A beam path or line may be defined by the location of the open MLC leaf and the location on the radiation imager where a pulse of radiation was detected. As shown in FIG. 4A, the radiation beamlet (401) intersects the target region (405). However, the radiation beamlet (403) does not intersect the target region (405). Since this radiation beamlet is not depositing any radiation to the target region, it may indicate an error in the radiotherapy system hardware or software, and a notification may be generated to inform the operator to pause radiation delivery. During and/or after a radiation delivery session, the radiotherapy system controller may combine the radiation beamlet trajectories to generate a “fluence cloud” of emitted radiation. Real-time monitoring of the radiation beamlet trajectories and/or radiation fluence cloud may provide additional safety checks and assurances that the radiotherapy system is operating as intended and radiation is being delivered to the target region(s). In some variations, the accumulated radiation beamlet trajectories may be used to determine a delivered dose to the patient (or phantom). This may be performed at the end of a radiation delivery session, and/or at specific intervals during the radiation delivery session. The delivered dose as determined at the end of a radiation delivery session may be used to adjust the radiation emitted at the next delivery session.

FIG. 4B depicts a flowchart representation of a method for tracing a radiation beamlet path or trajectory, and comparing the beamlet trajectory with the location of a target region or region of interest. Method (410) may comprise determining (402) the location of a region of interest, emitting (404) a radiation beamlet from a therapeutic radiation source where the beamlet is shaped by an opening of the beam-shaping assembly, acquiring (406) a radiation measurement of the emitted radiation beamlet using a radiation imager, determining (408) a path of the radiation beamlet from the radiation source to the radiation imager using the radiation measurement, and generating (411) a graphical representation that includes the radiation beamlet path and the location of the region of interest. The therapeutic radiation source may be a linac, the beam-shaping assembly may comprise an MLC, such as a bMLC, and the radiation imager may be located opposite the linac. Determining (402) the location of the region of interest (or target region) may comprise acquiring imaging data, such as CT imaging data, PET imaging data, or MR imaging data, that reveals the target region, and the location of the target region may be defined according to the coordinates of the radiotherapy system. Determining the location of the region of interest may also include defining a contour of the region of interest at the location identified in the imaging data. A contour may be a boundary of region of interest. It may be defined in three dimensions (i.e., a 3-D contour) or as a series of two dimensional slices (i.e., a 2-D contour) that sum together to form a 3-D volume. A 3-D contour may be depicted as a series of 2-D slices. In some variations, the contour of the region of interest may be defined on an anatomical image, such as a CT image and/or an MR image. For example, the contour of a region of interest may be an outline delineating the boundaries of the region of interest overlaid on top of an anatomical image. In some variations, the generated (411) graphical representation may comprise an anatomical image (2-D and/or 3-D), a contour of the region of interest depicted on the anatomical image, and a line representing the radiation beamlet path. As radiation is delivered, the graphical representation may include multiple lines for each of the emitted radiation beamlets, which may appear in real-time. A radiotherapy system operator may then, by visual inspection, determine whether the beamlet path intersects with the region of interest. Alternatively, or additionally, the radiotherapy system controller may be configured to automatically monitor the lines of the radiation beamlet paths to determine whether or not the lines intersect the region of interest. In some variations, the graphical representation may also include time stamps of each radiation beamlet, which may be based on the linac sensors and/or radiotherapy system controller subsystem which controls the timing of linac pulses. Optionally, method (410) may comprise generating (412) a notification of whether one or more radiation beamlet paths intersect the region of interest. In some variations, a notification may be generated if a threshold number of beamlet paths do not intersect the region of interest. Alternatively, or additionally, a notification may comprise a status indicator that has first visual appearance when the beamlet paths intersect the region of interest and a second visual appearance different from the first visual appearance when the number of beamlet paths that do not intersect the region of interest exceed a threshold number (e.g., more than zero, more than 5, more than 10, etc.). The generated notifications may comprise a visual notification and/or audio notification, as described above. The radiotherapy operator may then decide whether to proceed with radiation delivery based on the information provided in the notification and/or the graphical representation.

FIGS. 4C-4D depict flowchart representations of one variation of a method for determining a path of a radiation beamlet. In some variations, the methods of FIGS. 4C-4D may be used with method (410), e.g., for step (408). As described above, a radiation beamlet trajectory is a line extending from the radiation source toward the treatment area and ending at the radiation imager that may be defined by two points. For example, a radiation beamlet trajectory may be a line defined between the location of the open MLC leaf and a location on the radiation imager where a pulse of radiation was detected. Alternatively, or additionally, a radiation beamlet trajectory may be a line defined between the location of the therapeutic radiation source (e.g., linac) and a location on the radiation imager where a pulse of radiation was detected. The first point or location on the beamlet trajectory may be a location of a radiation beamlet before the beamlet interacts with the treatment area (e.g., couch) of the radiotherapy system, which may be in the vicinity of the linac and/or components of the beam-shaping assembly, such as the MLC. The second point or location on the beamlet trajectory may be a location of the radiation beamlet after it interacts with the treatment area, which may be a location on the radiation imager. FIG. 4C depicts one variation of a method of determining a radiation beamlet trajectory. Method (420) may comprise identifying (422) a first location of an opening in the beam-shaping assembly, identifying (424) a second location on the radiation imager based on the acquired radiation measurement, and determining (426) the radiation beamlet path by defining a line between the first location and the second location. Identifying (424) the second location on the radiation imager may comprise identifying a group of radiation imager detector elements (and/or radiation image pixels) that have a higher signal or intensity value than surrounding elements and/or a higher signal or intensity value than a predetermined threshold, and calculating the location of a center region of the group of detector elements (and/or radiation image pixels). The group of radiation imager detector elements may have the higher signal or intensity value at the same (or nearly the same) time as the linac emits a radiation beam pulse. The timestamp of the radiation beam pulse may be used to identify the MLC leaves open at that time and from that, identify the first location.

Alternatively, or additionally, identifying (424) the second location may use the method depicted in FIG. 4D. Method (430) may comprise selecting (434) the group of radiation image pixels that correspond to the open MLC leaf and calculating (436) a location of a centroid of the selected group of pixels. Optionally, before radiation is emitted, method (432) may comprise mapping (432) each leaf of the MLC to a group of detector elements (which may correspond to radiation image pixels) on the radiation imager. In some variations, this may include opening each MLC leaf individually and acquiring radiation measurements using the radiation imager. The detector elements or radiation image pixels that have a higher intensity may be mapped to that particular MLC leaf. The mapping between each MLC leaf and a group of corresponding detector elements or image pixels may be stored in a database memory of the radiotherapy system controller. Then, based on this mapping and the MLC leaf sensor data and/or MLC processor commands, selecting (434) the group of pixels may include retrieving from the database indexed by MLC leaf number, the corresponding group of pixels. Optional step (432) may be performed in a session that is separate from the radiation delivery session, for example, a calibration session or a system commissioning session.

While the examples described above are in the context of determining whether a radiation beamlet trajectory intersects a region of interest comprising a target region (e.g., tumor), it should be understood that similar methods may be used to determine whether a beamlet trajectory intersects an organ at risk and/or radiation-sensitive structure (i.e., radiation-avoidance regions). Monitoring whether radiation beamlet trajectories intersect radiation-avoidance regions may optionally comprise generating a notification if the number of beamlet trajectories (and/or radiation fluence amounts) intersecting a radiation-avoidance region exceeds a threshold number (and/or exceeds a threshold amount of radiation fluence).

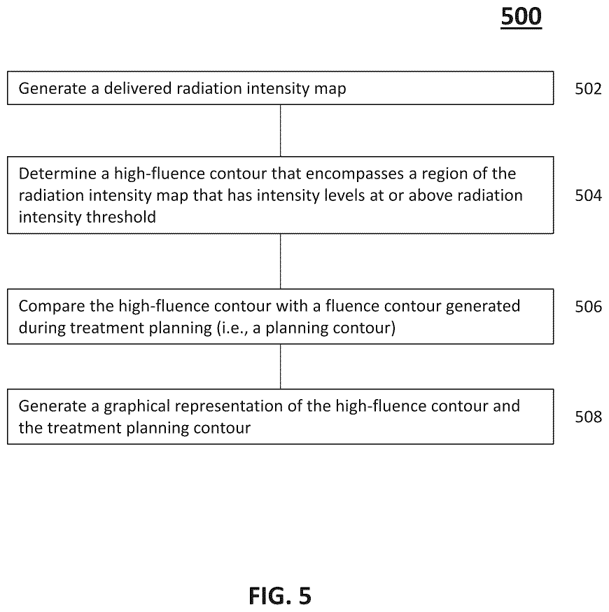

The radiation beamlet trajectory data acquired during a radiation delivery session may be combined to generate a radiation fluence distribution profile (which may be referred to as a delivered radiation intensity map). A radiation fluence distribution profile or radiation intensity map may represent the amount of radiation (i.e., radiation flux, radiation intensity) for various regions in space. Regions of the fluence distribution profile that have accumulated radiation fluence at or above a threshold may be defined as a “fluence cloud.” In some variations, the region (e.g., volume) enclosed by a high-fluence contour may be a fluence cloud, which comprises the substantially contiguous image pixels that have intensity values above the radiation intensity threshold. A fluence cloud may represent the region(s) that may have an elevated deposition of radiation. In some variations, the boundaries or contours of the fluence cloud may be a dose or fluence iso-contour. For a treatment session, it is desirable that this fluence cloud overlaps substantially (if not entirely) with the region of interest or target region. The spatial characteristics of the fluence cloud may be compared with the spatial location of the region of interest during a radiation delivery session. The amount of overlap between the fluence cloud and the region of interest may be monitored and a notification may be generated if the amount of overlap drops below a threshold. For example, if the percentage or proportion of the fluence cloud that overlaps with the region of interest is less than a percentage or proportion threshold, then a notification may be generated. One variation of a method is depicted in the flowchart of FIG. 5. Method (500) may comprise generating (502) a delivered radiation intensity map, where the intensity map is a spatial plot of radiation intensity (intensity incident on a pixel in the volume), determining (504) a high-fluence contour that encompasses a region of the radiation intensity map that has intensity levels at or above radiation intensity threshold, comparing (506) the high-fluence contour with a treatment planning contour, and generating (508) a graphical representation of the high-fluence contour and the treatment planning contour. Generating (502) a delivered radiation intensity map may comprise by forward-projecting (e.g., ray tracing) the emitted radiation beamlets across the treatment area.

Forward-projecting or ray tracing the emitted radiation beamlets may comprise calculating, for each pixel (or voxel) within the treatment area irradiated by the emitted radiation, a radiation fluence amount that passes through that pixel (or voxel). The forward projection process may be a simple “ray tracing” along the beamlet where an average fluence is assumed along each ray and the resulting fluences are simply added together on a pixel by pixel basis. Alternatively, or additionally, generating (502) a delivered radiation intensity map may comprise combining the radiation beamlet trajectories of the delivered radiation in an image, and summing the radiation fluence deposited at each pixel of the image. In some variations, the image may have a line for each beamlet trajectory and the intensity at each pixel may be a count of the number of beamlet trajectories that have crossed that pixel. In some variations, the count may be a sum product of the number of beamlet trajectories and the number of pulses in each beamlet, i.e., the count may be calculated by multiplying the number of beamlet trajectories and number of pulses in each beamlet and then summing these together. Improved accuracy of the fluence cloud may be realized by using imaging data (from the CT of the patient on the table) to consider attenuation of the beam as it traverses the patient as well as calculating contributions from beam scatter. While a radiation intensity threshold may be used, in some variations, the fluence cloud may comprise substantially contiguous image pixels that have intensity values greater than their surrounding pixels. The treatment planning contour may be the contour of a region of interest (ROI), and may be, for example, the contour of an internal target volume (ITV), planning target volume (PTV), biological targeting zone (BTZ), and the like. For each ROI contour, the treatment planning system can calculate an expected corresponding fluence contour. Comparing (506) the delivered high-fluence contours (e.g., boundaries of the fluence cloud, or a selected dose or fluence iso-contour) with the treatment planning ROI contour(s) and their corresponding planned fluence iso-contours may comprise determining the amount of overlap between the fluence cloud and the planning contour(s). The generated graphical representation (e.g., of step 508) may include a spatial plot that includes the treatment planning contour(s) and also the contour(s) of the fluence cloud(s). Optionally, the graphical representation may include visual indicia for areas of overlap between the treatment planning contour(s) and the fluence cloud contour(s), and optional different visual indicia for areas of the fluence cloud(s) that do not overlap with the treatment planning contour(s). In some variations, the spatial plot may be overlaid on an image that is registered to the plot, for example, an anatomical image (e.g., CT image and/or an MR image) and/or a biological or functional image (e.g., a PET image). The graphical representation may include text or visual symbols that indicate the amount of overlap and/or non-overlap between a fluence cloud and the treatment planning contours, for example, a percentage of the fluence cloud that overlaps with a contour of a region of interest. A visual or audio notification may be generated by the radiotherapy system controller if the amount of overlap is at or below a threshold. While the examples described herein relate to monitoring the radiation fluence delivered to regions of interest that are target regions (e.g., tumors), these methods may also be used to monitor the radiation fluence developed to regions of interest that are not target regions, such as organs at risk (OARs) or radiation-sensitive structures. In the context of monitoring radiation fluence to OARs or radiation-sensitive structures (i.e., non-target regions), a visual or audio notification may be generated by the radiotherapy system controller if the amount of overlap is at or above a threshold, and/or if the amount of radiation fluence within the treatment planning contours of an OAR exceed a threshold fluence amount.

Optionally, in some variations, the fluence cloud generated using any of the method described herein may be used to calculate the amount of radiation dose deposited into a patient or phantom using data about the radiation absorption and/or attenuation properties for different tissue types. Such data may be obtained from a CT image. For example, a CT image may be used to derive an attenuation coefficient for each portion (e.g., pixel or voxel) of the image. One variation of a method may comprise aligning the fluence cloud with the CT image, adjusting the fluence values of the fluence cloud according to the attenuation properties of the tissue that co-localizes with the fluence cloud, and calculating the radiation dose delivered to the tissue (and/or the entire patient) using the adjusted fluence values. The calculated delivered dose may be used to determine whether the radiation delivery session (i.e., treatment session) met clinical goals, and/or whether the radiation delivery for a following delivery session should be modified to compensate for any differences between the delivered dose and the desired dose.

### Methods for Managing Radiation Imager Artifacts Due to a Patient Implant

Any of the methods described herein for monitoring MLC function, monitoring linac function, determining one or more radiation beamlet trajectories and/or determining one or more radiation fluence clouds may optionally incorporate patient-specific information that may affect how radiation transmits through the patient. For example, the patient may have implants that comprise high-density materials that absorb more radiation than biological tissue (thus resulting in “dark pixels” on the radiation detector) and/or implants that comprise materials that scatter radiation or otherwise cause imaging artifacts on the radiation detector. Radiation scattering processes within the patient may also cause some pixels to have more signal intensity than what would be expected from simple geometrical ray tracing. Patient information regarding the location, geometry (size and shape), and radiation attenuation and/or scattering effects of the implant or other anatomical features such as bone or tissue may be provided to the treatment planning system (and then transmitted to the radiotherapy system) or otherwise stored in the radiotherapy system controller memory. The radiotherapy system controller may be configured to determine the location and geometry of the implant relative to the linac, MLC, and/or radiation imager for each position of the patient platform. In some variations, if it is determined that the radiation beamlet emitted from the linac with certain MLC leaves (or leaf) being open at a certain linac firing position would interact (e.g., intersect) with the implant, at least a portion of the radiation imager measurements may be excluded from the analysis. For example, when acquiring a radiation measurement to determine whether the MLC is operating correctly (e.g., method described and depicted FIG. 2A), the radiotherapy system controller may determine whether any portion of the radiation measurement (e.g., a group of image pixels or radiation detector elements) may contain “dark pixels” or otherwise measurement artifacts due to the patient's implant. The controller may use the location and geometry of the implant, along with the MLC configuration (e.g., leaf positions), and linac location to determine whether the data from any portion of the radiation imager (e.g., group of detector elements or imaging pixels) is impacted by the implant and may be excluded from the analysis and comparison. For example, if it is determined that the data from a group of detector elements would likely be affected by the implant, then the data acquired (204) from those detector elements may be ignored and excluded from the comparison (208) to determine whether the MLC motion characteristics are as expected. Similarly, in method (220), when the radiotherapy system controller is identifying pixels, pixel patterns, and mapping pixel patterns to MLC leaves (224-230), any pixels that may be “dark pixels” or otherwise contain noisy information due to the patient's implant(s) may be excluded from the pixel pattern identification and MLC leaf mapping steps. Pixel pattern identification (242) and pixel pattern mapping (244) of method (240) may similarly exclude any “dark pixels” or pixels that otherwise contain noisy information due to the patient's implant(s). Such “dark pixels” may also be excluded from analysis during methods that monitor linac function (e.g., method (300)), as well as the methods for determining one or more radiation beamlet trajectories and/or determining one or more radiation fluence clouds.

### Dose Monitoring

The methods described above may use imaging data from the radiation imager (e.g., MV detector) to confirm that the MLC, linac, and/or other components of the radiotherapy system are operating as expected. The operation and/or characteristics (e.g., motion characteristics) of these components as determined from the radiation imager measurements may be compared with the operation and/or characteristics as determined from system sensors and/or machine instructions from the radiotherapy system controller(s) as an additional verification that the radiotherapy system is functioning as intended. System sensors may include position sensors, motion sensors, orientation sensors, dose chambers (or any other radiation measurement tool), temperature sensors, pressure sensors, and the like. The imaging data from the radiation imager, alone or in combination with the system sensors, machine instructions, and/or anatomical imaging data (e.g., a CT image and/or any image with radiation attenuation data of anatomical structures) to calculate a delivered radiation dose distribution. By using the CT image data in conjunction with the radiation beamlet trajectory data, a delivered radiation dose distribution may be calculated without specialized (e.g., patient-specific) calibration of the radiation imager. The spatial characteristics of the delivered radiation dose distribution may be depicted on a 2-D or 3-D map where the spatial extent is represented on x-, y-, and z-axes (in 3-D), and the intensity of each pixel (or voxel) represents the amount of radiation dose delivered to that location. The delivered radiation dose map may be compared to a planned radiation dose map during a radiation delivery session, which may help the radiotherapy system operator know whether the treatment is on track. Alternatively, or additionally, the delivered radiation dose map may be compared to the planned radiation dose map after the radiation delivery session. Differences between the delivered radiation dose map and the planned radiation dose map may be adjust (i.e., adapt) the treatment for the next radiation delivery session (e.g., next treatment fraction). In some variations, the differences between delivered and planned dose maps may be used in offline adaptation methods to update the treatment plan for the next radiation delivery session.

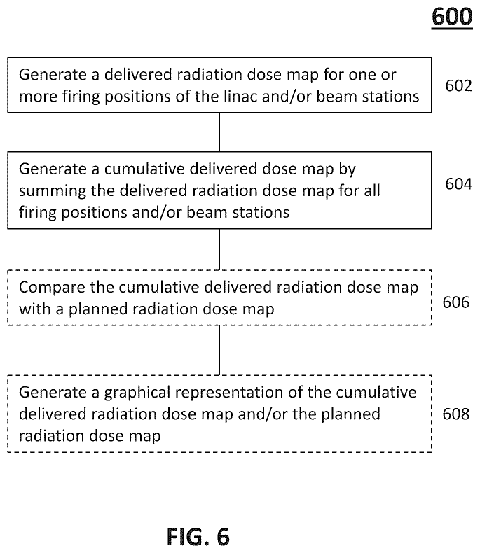

FIG. 6 depicts a flowchart representation of one variation of a method for generating a cumulative delivered radiation dose distribution or map. Method (600) may comprise generating (602) a delivered radiation dose map for one or more firing positions of the linac and/or beam stations (e.g., patient platform positions) and generating (604) a cumulative delivered radiation dose map by summing the delivered radiation dose map for all firing positions and/or beam stations. In one variation, generating (602) the delivered radiation dose map for one or more firing positions and/or beam stations may comprise forward-projecting (e.g., ray tracing) MLC patterns and/or radiation beamlet trajectories by calculating the radiation deposition along the beamlet trajectory using CT image data. Alternatively, or additionally, generating (602) the delivered radiation dose map for one or more firing positions and/or beam stations may comprise back-projecting measurements from the radiation imager (e.g., measurements from the MV detector) using inverse attenuation and CT image data. The CT image data may be acquired as part of radiation therapy treatment planning and optionally, at the beginning of the radiation delivery session and/or during the radiation delivery session. Method (600) may optionally comprise comparing (606) the cumulative delivered radiation dose map with a planned radiation dose map and generating (608) a graphical representation of the cumulative delivered radiation dose map and/or the planned radiation dose map. In one variation, the delivered radiation dose map may be superimposed on the planned radiation dose map. Areas of non-overlap may be shaded or otherwise highlighted with one color or pattern, while optionally, overlap areas may be shaded with a different color or pattern. In some variations, one or more of the delivered radiation dose map and/or the planned radiation dose map may be superimposed on an anatomical image of the patient (e.g., CT image, MR image).

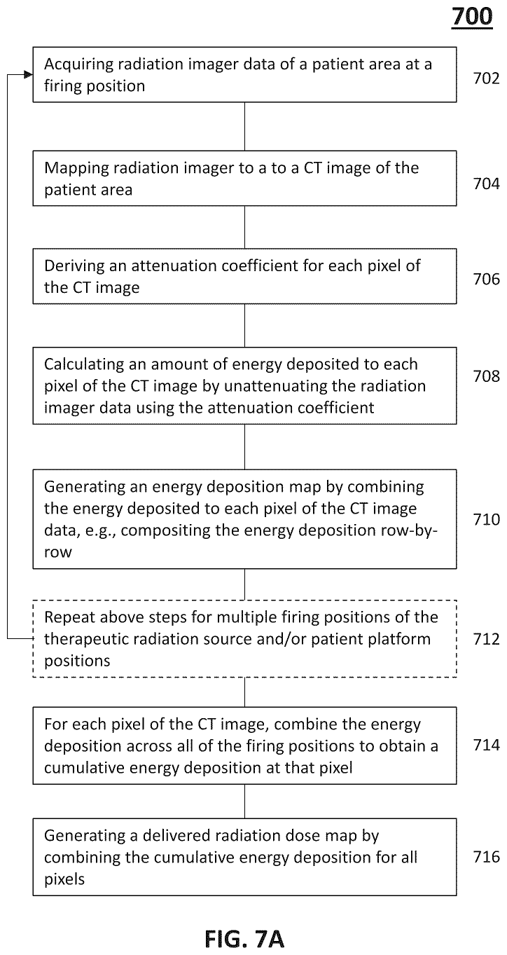

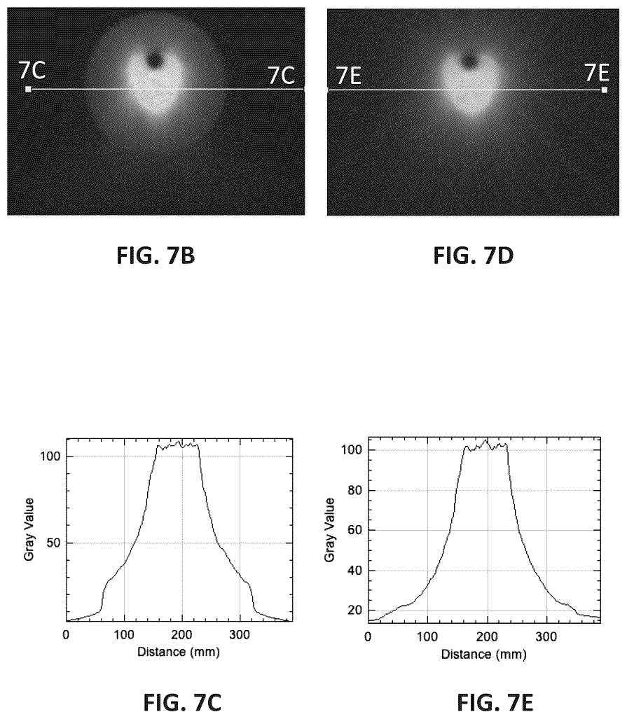

FIG. 7A depicts one variation of a method for generating a delivered radiation dose map by back-projecting radiation measurements from the radiation imager (i.e., MV detector) and unattenuating (i.e., reversing the attenuation of radiation by intervening tissue) based on CT image data. Back-projection is a reconstruction method that comprises calculating, for each projection angle around a patient, the radiation emitted by the radiation source that would result in the radiation imager measurement, using known geometry. This may include calculating the amount of radiation absorbed (i.e., attenuated) by the patient tissue for each pixel (or voxel) along the radiation beamlet trajectory. Some variations of back-projection may comprise compensating for the attenuation of the radiation by patient tissue to determine the amount of radiation emitted by the radiation source. Reversing the attenuation of (or un-attenuating) the radiation may comprise adding back the radiation that was absorbed by patient tissues. Different tissue types have different absorption and/or attenuation properties, and in some variations, the attenuation properties of tissue along a radiation beamlet trajectory may be calculated from a CT image. The data from a CT image may be used to derive an attenuation coefficient for each portion (e.g., pixel or voxel) of the CT image. FIG. 7A depicts one variation of a method of calculating a dose map using radiation imager data of and a CT image. Method (700) may comprise acquiring (702) radiation imager data of a patient area resulting from emitting radiation at a therapeutic radiation source firing position, mapping (704) radiation imager data to a CT image of the patient area, deriving (706) an attenuation coefficient for each pixel of the CT image, calculating (708) an amount of energy deposited to each pixel of the CT image by unattenuating the radiation imager data using the attenuation coefficient, and generating (710) an energy deposition map by combining the energy deposited to each pixel of the CT image data. In some variations, mapping radiation imager data to a CT image may include aligning the radiation imager to the CT image. Generating (710) the energy deposition map may comprise compositing the energy deposition on a row-by-row basis for the CT image. Method (700) may comprise repeatedly calculating energy deposition maps in accordance with steps (702-710) for one or more firing positions of the therapeutic radiation source and radiation imager (which may be movable or rotatable while still having a fixed relative position to each other) and/or one or more positions of the patient platform. For example, energy deposition maps may be calculated for all firing positions around a patient (e.g., firing angles ranging from 0° to 359°, continuously and/or at discrete circumferential locations) from which therapeutic radiation was emitted. Some methods may comprise generating energy deposition maps for all firing angles at all planned patient platform locations (i.e., beam stations). Method (700) may comprise combining (714), for each pixel of the CT image, the energy deposition across all of the firing positions to obtain a cumulative energy deposition at that pixel, and generating (716) a delivered radiation dose map by combining the cumulative energy deposition for all pixels of the CT image. Optionally, method (700) may comprise generating a graphical representation of the delivered radiation dose map with treatment planning contours overlaid on the dose map.

In some variations, method 700 may comprise calibrating the radiation imager before it is used in a radiation delivery session for generating a delivered radiation dose map. One variation of calibration the radiation imager may comprise determining, from CT imaging data, the percentage of each beamlet path that traverses through air, bone and soft tissue, and generating one or more radiation imager calibration tables based on relative percentages of each kind of tissue as well as overall path length. The information in the generated calibration table(s) may provide a mapping between radiation imager measurements taken at different angles with the energy spectrum of the beamlet emitted along that angle. This may facilitate a more accurate measurement of the beam exit fluence for the back projection process.

In some instances, the contribution of scattered radiation to the radiation imager imaging data may be significant. Optionally, some methods may comprise generating a scatter kernel that represents an estimate of the scatter component. The scatter kernel may be generated based on the radiation beam energy and the attenuation along the beamlet path. Alternatively, or additionally, methods of generating a scatter kernel may include solving the linear Boltzmann transport equation and/or Monte Carlo simulations. The scatter kernel may be used to determine the amount of the radiation detected by the imager that is the result of scatter. Once the scattered radiation intensity is known, it may be subtracted from the radiation imager signal to more accurately determine the directly attenuated signal.

In one example, a radiotherapy treatment plan may be generated by a treatment planning system (TPS) for a head and neck target. FIGS. 7B-7C depict the simulated radiation dose distribution according to the treatment plan and FIGS. 7D-7E depict the reconstructed radiation dose distributions generated from radiation imager measurement data of radiation delivery of the treatment plan by a radiotherapy system. The intensity of each pixel in the dose distributions of FIGS. 7B and 7D correlates to the amount of radiation delivered to that corresponding pixel (or voxel) in space. The planned radiation dose generated by a processor or controller of the TPS depicted in FIGS. 7B-7C are simulated radiation dose distributions that represent the radiation dose as if radiation were delivered according to the radiotherapy treatment plan. FIG. 7B is a simulated radiation dose map (i.e., generated by the TPS) and FIG. 7C is a plot of the radiation dose values along the line 7C-7C. In some variations, the simulated image of FIG. 7B may be generated by simulating the insertion of an object or phantom into the field of view for dose calculation. The object or phantom can be modeled or simulated to be made of a very low density material, e.g., having a density that is similar or close to that of air. After the TPS generates the treatment plan, the radiotherapy system may be operated to emit or deliver radiation according to the treatment plan. The delivered radiation may be measured using the radiation imager, and the method (700) of FIG. 7A may be used to generate a delivered radiation dose distribution (e.g., map) from the radiation imager data. In this example, the planned radiation was delivered using a radiotherapy system without a phantom on the patient platform (i.e., it was an “air delivery”). An air delivery of radiation eliminates (or greatly reduces) the attenuation effect and so attenuation correction is optional in the dose reconstruction. The delivered radiation was measured by the radiation imager (e.g., MV detector) of the radiotherapy system, which is located across from the linac. FIG. 7D depicts an image of the delivered dose reconstructed from the radiation imager measurements. The reconstructed dose was generated from the radiation imager measurements using the method (700), as explained and depicted above in FIG. 7A. In this example, because the dose was delivered without a phantom (e.g., air delivery), there was no attenuation correction. FIG. 7E is a plot of radiation dose values taken along the line 7E-7E in FIG. 7D. A comparison of the dose image of FIG. 7B with the dose image of FIG. 7D demonstrates that qualitatively, the dose image reconstructed from the radiation imager data using the back-projection method (700) is similar to the dose image derived from the planning dose calculated (or simulated) by the TPS. The general shape, size, and position of the delivered radiation dose are similar between FIG. 7B (simulated dose) and FIG. 7D (calculated from radiation imager measurement data). This similarity is also represented in the radiation dose profiles in the plots depicted in FIG. 7C (simulated dose) and FIG. 7E (calculated from radiation imager measurement data). The dose value (in Gy) attains a similarly high value in both the imager-measured dose and the TPS simulated calculated dose, and they both have a similar curve shape. In this example, the edges of the simulated dose in FIG. 7C are sharper than the edges of the measured dose in FIG. 7E.

In some variations, the delivered radiation dose map may be evaluated to determine how well it adhered to the planned radiation dose map. One method of evaluating the delivered radiation dose map against the planned radiation dose map may comprise a gamma [symbol] evaluation. A gamma evaluation may comprise calculating a gamma [symbol] metric value for a pre-determined distance-to-agreement criterion (C_{DTA}) and a predetermined percent dose different criterion (C_{DD}), and determining whether the calculated gamma metric value meets a pre-determined threshold. Calculating the gamma [symbol] metric value may comprise calculating, for each pixel on the delivered radiation dose map, a distance-to-agreement value (DTA) to the planned radiation dose map and a percent dose difference (DD) to the planned radiation dose, where the gamma metric value is given by:

$$γ = \sqrt{{( \frac{DTA}{{C}_{DTA}} )}^{2} + {( \frac{DD}{{C}_{DD}} )}^{2}}$$

If the gamma metric value meets the pre-determined threshold (e.g., [symbol] <1), the delivered radiation dose map may be considered sufficiently similar to the planned radiation dose map. Otherwise, if the gamma metric value does not meet the threshold (e.g., [symbol] ≥1), a notification may be generated by the radiotherapy system controller. The clinician and/or operator may be prompted to adapt and/or otherwise adjust the radiotherapy treatment plan and/or delivery for the next treatment session. In some variations, a gamma evaluation may include evaluating whether the delivered dose distribution meets dose-difference (DD) and/or distance-to-agreement (DTA) criteria (e.g., 3%/3 mm) for multiple points on the dose distribution as compared to a reference dose distribution (e.g., the planned dose distribution).

Alternatively, or additionally, the delivered radiation dose map may be evaluated by calculating a delivered dose volume histogram (DVH) for each region of interest from the delivered radiation dose map, and determining whether the calculated DVH is within an acceptable dose distribution range represented by a bounded DVH calculated at the time of treatment planning. A DVH is a plot that represents the proportion of a region of interest that receives a certain amount of radiation. A DVH plot may have a first axis representing a volume proportion (from 0% to 100%) and a second axis that represents the amount of dose per volume proportion. A bounded DVH may have a first curve (i.e., a maximum curve) representing an acceptable upper limit to the amount of radiation delivered to a structure and a second curve (i.e., a minimum curve) representing an acceptable lower limit to the amount of radiation to the structure. In one variation, if the number or percentage of points on the delivered DVH curve are within the bounds of the bDVH meet or exceed a threshold, then the delivered radiation dose map may be considered sufficiently similar to the planned radiation dose map. Otherwise, if the number or percentage of points on the delivered DVH curve are within the bounds of the bDVH are below a threshold, a notification may be generated by the radiotherapy system controller. The clinician and/or operator may be prompted to adapt and/or otherwise adjust the radiotherapy treatment plan and/or delivery for the next treatment session. Optionally, some variations may comprise generating a graphical representation that includes the delivered DVH overlaid with the planned DVH. In some variations, the graphical representation may include visual indicia indicating whether the percentage of points meet or exceed a predetermined threshold.

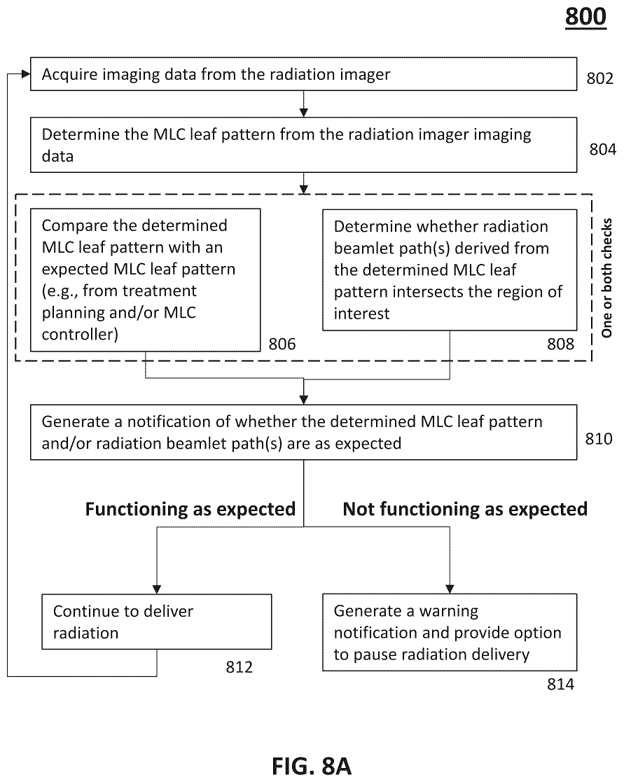

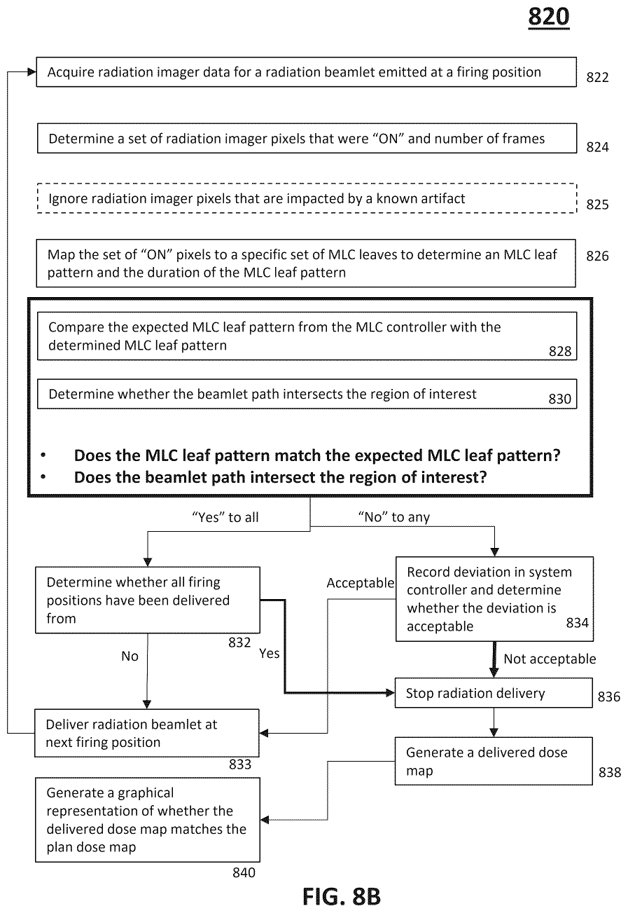

One or more of the methods described herein may be used alone or in combination to monitor radiation delivery in real-time. These methods may provide information about whether the components of the radiotherapy system are operating as expected and/or provide information about whether the radiation being delivered is according to the radiotherapy treatment plan. These methods may be included as part of a quality assurance (QA) session where radiation is delivered in the absence of a patient, and the radiation imager (e.g., MV detector) acquires radiation measurements that may be used to determine the quality of the treatment plan and/or the operation of the radiotherapy system components. FIG. 8A depicts one variation of a method for monitoring radiation delivery for either a patient treatment session or a QA session (in the absence of a patient). Method (800) may comprise acquiring (802) imaging data from the radiation imager (e.g., MV detector) as the therapeutic radiation source is emitting radiation, determining (804) the MLC leaf pattern from the radiation imager imaging data, and comparing (806) the determined MLC leaf pattern with an expected MLC leaf pattern (e.g., from treatment planning and/or MLC controller). Alternatively, or additionally, method (800) may comprise determining (808) whether radiation beamlet path(s) derived from the determined MLC leaf pattern intersects the region of interest. Any of the methods described above may be used for steps 806 and 808. Method (800) may comprise generating (810) a notification of whether the determined MLC leaf pattern and/or radiation beamlet path(s) are as expected. If the MLC leaf pattern and/or radiation beamlet path(s) are determined to be as expected (e.g., according to the treatment plan and/or commands from the radiotherapy controller), method (800) may comprise continuing to deliver (812) radiation and monitoring the delivered radiation (802-810). If the MLC leaf pattern and/or radiation beamlet path(s) are determined to be different from what was expected (e.g., according to the treatment plan and/or commands from the radiotherapy controller), method (800) may comprise generating (814) a warning notification and providing an option to the operator to pause radiation delivery.

FIG. 8B depicts a flowchart representation of one variation of a method for monitoring radiation delivery for either a patient treatment session or a QA session (in the absence of a patient). Method (820) may comprise acquiring (822) radiation imager (e.g., MV detector) data a radiation beamlet emitted at a firing position of a therapeutic radiation source, determining (824) a set of MVD detector pixels (e.g., detector elements) that were “ON” and number of frames, and mapping (826) the set of “ON” pixels to a specific set of MLC leaves to determine an MLC leaf pattern and the duration of the MLC leaf pattern. Optionally, in some variations, method (820) may comprise ignoring (825) or otherwise removing radiation imager pixels that are impacted by a known artifact (e.g., resulting from a patient implant) so that they are not used to determine the MLC leaf pattern. Determining whether a detector pixel or element is “ON” may comprise comparing the detector pixel intensity with an intensity threshold and if the intensity meets or exceeds the threshold, that pixel may be considered an “ON” pixel. In some variations, the intensity threshold may be the same for all detector pixels or elements, while in other variations, the intensity threshold may be different for each detector pixel or element. For example, when a patient or phantom is located on the platform, some detector elements may be obscured while others are not. In some variations, the intensity threshold for a detector pixel or element may be determined using a CT image of the patient or phantom, which may provide information about whether detector elements may be obscured by the patient or phantom. Detector elements that may be obscured by the patient or phantom as indicated by the CT image may have a lower intensity threshold (to be considered “ON”) than detector elements that are not obscured. Method (820) may comprise comparing (828) the expected MLC leaf pattern from the MLC controller with the determined MLC leaf pattern (optionally, with any adjustments for known CT artifacts) and/or optionally determining (830) whether the beamlet path intersects the region of interest (optionally, with any adjustments for known CT artifacts). If the MLC leaf pattern matches the expected MLC leaf pattern and the radiation beamlet path intersects the region of interest, method (820) may comprise determining (832) whether all firing positions have been delivered from, e.g., according to the treatment plan and/or the radiotherapy system controller instructions. If not, method (820) may comprise delivering (833) a radiation beamlet at the next firing position and continuing to monitor the MLC leaf pattern and beamlet path (i.e., steps 822-830). If all firing positions have been delivered from, method (820) may comprise stopping (836) radiation delivery.

If the MLC leaf pattern does not match the expected MLC leaf pattern and/or the radiation beamlet path does not intersect the region of interest, method (820) may comprise recording (834) the deviation in the radiotherapy system controller and determining whether the deviation is acceptable. In some variations, the determination as to whether the deviation is acceptable may be performed by a controller of the radiotherapy system, the operator, and/or both. For example, if the number of MLC leaves that are mismatched are below a threshold number of MLC leaves and/or if the radiation beamlet trajectory misses the region of interest by a distance that is below a threshold distance, the deviation may be considered acceptable by the radiotherapy system controller, and then confirmed by the operator. Alternatively, or additionally, some methods may comprise generating a graphical representation indicating the deviation and a quantification of the deviation (which may be textual information and/or pictorial information such as a plot) and receiving operator input as to whether the deviation is acceptable. If the deviation is within an acceptable range, method (820) may comprise delivering (833) a radiation beamlet at the next firing position and continuing to monitor the MLC leaf pattern and beamlet path (i.e., steps 822-830). If the deviation is not in the acceptable range, method (820) may comprise stopping (836) radiation delivery.

In some variations, method (820) may comprise generating (838) a delivered dose map or distribution and generating (840) a graphical representation of whether the delivered dose map matches the plan dose map. Generating (838) the delivered dose map and generating (840) the graphical representation may occur after radiation delivery is stopped (836), and/or may occur one or more times during radiation delivery. The delivered dose map may be generated using radiation imager and/or MLC position sensor data and/or CT imaging data, according to any of the one or more methods described herein. The comparison of the delivered dose map and the plan dose may use the gamma analysis and/or bDVH analysis described above. The generated graphical representation may include textual or graphical indicators of whether the delivered dose map matches the planed dose map. Examples of text may include “ACCEPTABLE” or “WITHIN-RANGE”, “UNACCEPTABLE” or “OUT-OF-RANGE”. Examples of graphical indicators may include colors (e.g., green for an acceptable dose map match, red for an unacceptable dose map match), shapes (e.g., a check-mark for an acceptable dose map match, a “X” for an unacceptable dose map match, and/or animations (e.g., a spinning graphic for an acceptable dose map match, a flashing for an unacceptable dose map match). In some variations, the graphical representation may have an anatomical plot (e.g., from a CT image or MR image), outlines of the treatment planning contours on the anatomical plot, and an outline of the region(s) of the delivered radiation dose map that have elevated dose values (e.g., an outline of high-dose or high-fluence regions). This graphical representation may be output to a display device for visual inspection by the system operator and/or clinician, where it may be readily discernible whether the high-dose regions of the delivered radiation dose map overlap substantially (or sufficiently) with the contours of the region of interest and/or contours of the target region(s).

Measurements from the radiation imagers (e.g., MV detector) may be used for evaluating the quality of treatment plans and confirming that a particular fluence map is able to be delivered with a certain level of accuracy by the radiotherapy system. This QA process may be referred to as a treatment plan QA process. During a treatment plan QA process, the planned fluence map generated by the radiotherapy treatment planning system (which may be integrated, or otherwise in communication with, the radiotherapy system) may be converted into a series of radiotherapy machine instructions. In some variations, the planned fluence map may comprise the planned fluence map for each of a plurality of beam stations, the planned fluence map for each of a plurality of shuttle passes, and/or the planned fluence map for the entire QA session. The radiotherapy machine instructions may specify the states of components of the radiotherapy system, such as MLC leaf configurations for a particular linac firing position for a particular patient platform position, where sequentially executing these component states will result in the delivery of radiation that closely matches the planned fluence map. The radiotherapy machine instructions may comprise a plurality of control points, where each control point includes the state of radiotherapy system components (e.g., MLC leaf configuration, jaw opening width, linac pulse sequence, linac firing position or angle, patient platform position, gantry rotation speed) at a series of time points during a radiation delivery session. For example, radiotherapy machine instructions may specify the configuration or state of the radiotherapy system components at a plurality of beam stations (e.g., patient platform or couch positions) at one or more couch shuttle passes through the treatment area (e.g., linac radiation beam path). The radiotherapy system may be configured to deliver radiation by following these radiotherapy machine instructions, one step at a time (i.e., sequentially in time), one control point at a time. The radiation imager may record the radiation emitted at each beam station, during an entire shuttle pass (which may be the accumulation of the radiation emitted at all of the beam stations that comprise a shuttle pass), and/or during an entire QA session (which may be the accumulation of the radiation emitted at all of the shuttle passes). The recorded radiation may be used to calculate one or more delivered fluence maps (e.g., a fluence map for each of a plurality of beam stations, a fluence map for each of a plurality of shuttle passes, a fluence map for the entire QA session), which may then be compared with their corresponding planned fluence maps. For example, the delivered fluence map for the entire QA session may be compared with the planned fluence map for the entire session. Alternatively, or additionally, the delivered fluence map for each beam station or each shuttle pass may be compared with the planned fluence map for each of the same beam stations or shuttle passes (respectively). If the difference between the delivered fluence map(s) and the planned fluence map(s) are below a threshold (e.g., the difference is within an acceptable range), then the planned fluence map(s) and/or radiotherapy system operation have passed the QA evaluation. If the difference between the delivered fluence map(s) and the planned fluence map(s) are above a threshold (e.g., the difference is outside an acceptable range), then the planned fluence map(s) and/or radiotherapy system operation have failed the QA evaluation. Some variations may comprise generating a notification to the operator as to whether a planned fluence map(s) has passed or failed, and in the event of a failure, the notification may include suggestions for troubleshooting the system and/or generating a new treatment plan with a new planned fluence map.

In some variations, the planned fluence map may be the fluence map generated the treatment planning that is intended to be delivered to the patient. The QA methods described herein may be evaluating the quality of this planned fluence map for patient treatment. In some variations, a QA fluence map may be generated from the planned fluence map by scaling, rotating, and/or shifting the planned fluence map intended for delivery to the patient. The radiation delivered during a QA session may be based on the QA fluence map instead of the planned fluence map. While the examples described herein discuss QA methods based on the planned fluence map, it should be understood that in some variations, the same QA methods may be used for a QA fluence map instead of a treatment planned fluence map.

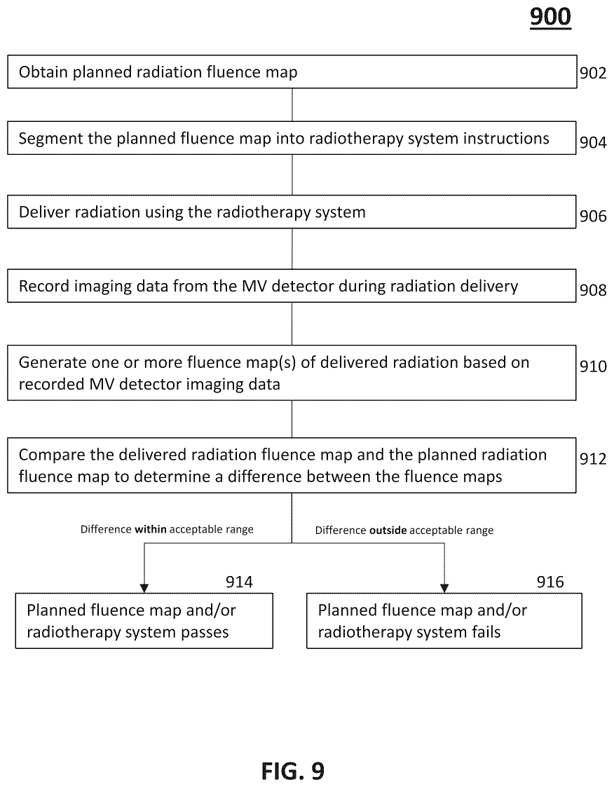

One variation of a method for evaluating a fluence map (e.g., a treatment planned fluence map and/or a QA fluence map) and the ability of a radiotherapy system to deliver the fluence map during a QA session is depicted in FIG. 9. Method 900 may comprise obtaining 902 a planned radiation fluence map (or a QA radiation fluence map), segmenting 904 the planned fluence map into radiotherapy system instructions, delivering 906 radiation using the radiotherapy system based on the radiotherapy system instructions, recording 908 imaging data from the MV detector during radiation delivery, generating 910 one or more fluence map(s) of delivered radiation based on record MV detector imaging data, and comparing 912 the one or more delivered radiation fluence map(s) and the planned radiation fluence map to determine a difference between the fluence maps. If the difference is within an acceptable range, the planned fluence map and/or radiotherapy system passes 914 the evaluation. If the difference is outside an acceptable range, the planned fluence map and/or radiotherapy system fails 916 the evaluation.

In some variations, a fluence map may comprise a plurality of radiation fluence sinograms. A radiation fluence sinogram is a plot of the radiation fluence across the total number of MLC leaves and the total number of linac firing angles. As an example, the x-axis of a radiation fluence sinogram may be an index of the MLC leaves (for example, leaf index 1 to leaf index 64 for a 64-leaf MLC) and the y-axis may be an index of the possible firing angles (for example, firing angle index 1 to firing angle index 50 for a total of 50 firing angles). There may be a plurality or a stack (e.g., along a z-axis) of radiation fluence sinograms that together comprise a fluence map. Each beam station and/or couch position may have its own radiation fluence sinogram, and a fluence map for a treatment plan may be the sum of the radiation fluence sinograms for all of the beam stations and/or couch positions. A planned fluence map may be generated by a treatment planning system, where the planned fluence map is intended to deliver a prescribed radiation dose to a target region while limiting the radiation exposure to surrounding tissue (including, for example, OARs). In some variations, method 900 may be used with a planned fluence map intended for delivery to a patient (if it passes the QA evaluation). In some variations, method 900 may be used with a QA fluence map instead of a planned fluence map (i.e., intended for delivery to a patient). The QA fluence map may be derived from the planned fluence map. For example, deriving a QA fluence map may comprise rotating and/or shifting and/or translating the planned fluence map.

Recording 908 imaging data from the MV detector during delivery may comprise storing the imaging data along with the radiotherapy machine instructions at the imaging data was collected. For example, recording the imaging data may comprise storing, in a memory of the radiotherapy system controller, the time at which the imaging data was acquired, the corresponding patient platform position (or beam station), the radiation source firing angle (e.g., gantry angle), and linac pulse(s). Generating 910 a delivered radiation fluence map that is cumulative across the entire QA session may comprise accumulating the MV detector images acquired from the same beam station, the same firing angle, and summing these MV detector images across all beam stations and firing angles. In some variations, generating 910 a fluence map of delivered radiation may comprise scaling and rebinning each MV detector image based on the geometry of the MV detector into the same sinogram space as the sinograms that comprise the planned fluence map. For example, generating a fluence map of delivered radiation may comprise scaling the MV detector image(s) using a pre-calibrated image intensity-to-fluence conversion factor and rebinning the image(s) based on the MV detector geometry. Comparing 912 the delivered fluence map with the planned fluence map may comprise comparing the cumulative delivered fluence map (i.e., a fluence map that represents all of the radiation delivered during the QA session) with the planned fluence map corresponding to an entire treatment session. Alternatively, or additionally, comparing 912 the delivered fluence map with the planned fluence map may comprise comparing a plurality of delivered fluence maps with a corresponding plurality of corresponding planned fluence maps, for example, comparing a delivered fluence map with at a beam station with the planned fluence map at the same beam station, and/or compared a delivered fluence map for a shuttle pass with the planned fluence map for the same shuttle pass.

Method 900 may optionally comprise generating a notification to the user that contains information about whether the fluence map and/or radiotherapy system passes or fails the evaluation. In some variations, the notification may comprise information pertaining to the operation and function of the radiotherapy system components, such as the linac, rotatable gantry, MLC (including other radiation beam-shaping components), and patient platform (i.e., couch) position during the QA session. For example, if any component malfunctioned or performed out of its specification during the QA session radiation delivery, the notification may include that information, which may help the user determine whether the radiotherapy system needs to be repaired, calibrated, or otherwise adjusted. If there was no malfunction of the radiotherapy system during radiation delivery of method 900, and the delivered radiation fluence map is significantly different from the planned radiation fluence map, the notification may have a status indicator informing the user that the radiotherapy system operated as expected (e.g., a green color status) and a text indicator to suggest that the user initiate alternative approaches to troubleshooting, including, but not limited to, debugging the software elements of the radiotherapy system and/or treatment planning system.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described systems and methods can be implemented in any of numerous ways. For example, at least some methods of the present technology may be implemented using hardware, firmware, software, or a combination thereof. When implemented in firmware and/or software, the firmware and/or software code can be executed on any suitable processor or collection of logic components, whether provided in a single device or distributed among multiple devices.

In this respect, various aspects described herein may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods disclosed herein need not reside on a single computer or processor but may be distributed in a modular fashion amongst a number of different computers or processors to implement various aspects of the inventions disclosed herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in different variations.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the acts performed as part of the method may be ordered in any suitable way. Accordingly, various methods may be constructed in which acts are performed in an order different than illustrated, which may include performing some acts simultaneously, even though shown as sequential acts in illustrative examples.

1. A method for verifying multi-leaf collimator (MLC) functionality during a radiation delivery session, the method comprising:
generating a multi-leaf collimator (MLC) pattern during the radiation delivery session, wherein the MLC pattern designates an expected MLC leaf location for each leaf of the MLC, wherein the MLC is located in front of a radiation source;
emitting radiation from a radiation source, wherein the emitted radiation is shaped by the MLC;
acquiring a radiation measurement of the emitted radiation using a radiation imager located opposite the radiation source;
determining a location of an MLC leaf using the radiation measurement;
comparing the determined MLC leaf location with the expected MLC leaf location; and
generating an MLC leaf location notification.

2. The method of claim 1, further comprising:
calculating a time duration of the MLC leaf at the determined MLC leaf location using the radiation measurement;
comparing the calculated time duration with an expected time duration of the MLC leaf at the expected location; and
generating an MLC dwell time notification based on the comparison between the calculated time duration and the expected time duration.

3. The method of claim 1, further comprising:
calculating an intensity of the emitted radiation using the radiation measurement;
comparing the calculated intensity with an expected radiation intensity; and
generating an intensity notification based on the comparison between the calculated intensity and the expected radiation intensity.

4. The method of claim 3, wherein the emitted radiation comprises one or more radiation pulses and the expected radiation intensity comprises an expected number of radiation pulses, wherein calculating the intensity of the emitted radiation comprises calculating a number of radiation pulses using the radiation measurement, and wherein comparing the calculated intensity with the expected radiation intensity comprises comparing the calculated number of radiation pulses with the expected number of radiation pulses.

5. The method of claim 1, wherein no patient is present when the radiation is emitted.

6. The method of claim 1, wherein a patient is present when the radiation is emitted.

7. The method of claim 1, wherein the radiation measurement comprises intensity values of each pixel of the radiation imager, and wherein determining a location of the MLC leaf comprises identifying pixels of the radiation imager that had an intensity over a threshold level and mapping the identified pixels to the MLC leaf and its location.

8. The method of claim 7, wherein the location of the MLC leaf is determined based on a pattern of pixels having intensity values below the threshold level.

9. The method of claim 7, wherein the location of the MLC leaf is determined based on a pattern of pixels having intensity values at or above the threshold level.

10. The method of claim 7, wherein the radiation measurement comprises one or more frames, wherein each frame has intensity values of each pixel of the radiation imager over a pre-determined acquisition time period, and wherein determining the location of the MLC leaf further comprises calculating a dwell time that the MLC leaf is at the determined location based on a number of frames in which a set of pixels have an intensity over the threshold value.

11. The method of claim 10, further comprising comparing the calculated dwell time with an expected dwell time, and wherein generating a notification further comprises generating a notification if the calculated dwell time is different from the expected dwell time.

12. A method for verifying radiation beamlet delivery to a region of interest, the method comprising:
emitting a radiation beamlet from a radiation source, wherein the emitted radiation beamlet is defined by an opening of a multi-leaf collimator (MLC) located in front of the radiation source;
acquiring a radiation measurement of the emitted radiation beamlet using a radiation imager located opposite the radiation source;
determining a path of the radiation beamlet from the radiation source to the radiation imager using the radiation measurement; and
generating a graphical representation that includes the radiation beamlet path and a location of a region of interest.

13. The method of claim 12, further comprising generating a notification if the radiation beamlet path does not intersect the region of interest.

14. The method of claim 13, wherein the region of interest is defined by a boundary and the method comprises generating a notification if the radiation beamlet path does not cross the boundary of the region of interest.

15. The method of claim 12, wherein no patient is present when the radiation beamlet is emitted.

16. The method of claim 12, wherein a patient is present when the radiation beamlet is emitted.

17. The method of claim 16, wherein the region of interest comprises an internal tumor volume (ITV).

18. The method of claim 16, wherein the region of interest comprises a biology tracking zone (BTZ).

19. The method of claim 12, wherein determining the path of the radiation beamlet comprises identifying a first location of the MLC opening, identifying a second location on the radiation imager based on the acquired radiation measurement, and determining the radiation beamlet path by defining a line between the first location and the second location.

20. The method of claim 19, wherein the graphical representation includes an outline of a boundary of the region of interest and a line that represents the radiation beamlet path.

21. A method for verifying a delivered fluence, the method comprising:
emitting radiation beamlets from a radiation source;
generating a delivered radiation intensity map by forward-projecting the emitted radiation beamlets, wherein the radiation intensity map comprises a spatial plot of radiation intensities;
defining a high-fluence contour that encompasses a region of the delivered radiation intensity map that has intensity levels at or above a radiation intensity threshold;
determining a contour difference between the high-fluence contour and a treatment planning contour by registering the treatment planning contour with the delivered radiation intensity map; and
generating a graphical representation that includes the high-fluence contour and the treatment planning contour overlaid on the delivered radiation intensity threshold, and a notification of the contour difference.

22. The method of claim 21, wherein the emitted radiation beamlets comprise an irradiation field and wherein generating the delivered radiation intensity map comprises calculating emitted fluences based on the openings of the MLC, radiation source pulses, and radiation source locations, and plotting the emitted fluences over the irradiation field.

23. The method of claim 21, wherein the radiation intensity map is a 3D spatial plot.

24. The method of claim 21, wherein the radiation intensity map is a 2D spatial plot.

25. The method of claim 21, wherein defining a high-fluence contour comprises identifying a pixel of the radiation intensity map having a higher intensity value than the other pixels in the radiation intensity map, calculating a threshold intensity value by calculating a percentage of the highest intensity value, identifying a group of pixels in the radiation intensity map that have intensity values at or above the threshold intensity value, and defining the high-fluence contour by outlining a perimeter of the group of pixels.

26. The method of claim 25, wherein the percentage of the highest intensity value is 70% or more.

27. The method of claim 25, wherein the percentage of the highest intensity value is 80% or more.

28. The method of claim 21, wherein defining a high-fluence contour comprises calculating a center of mass of the radiation intensity map using intensity values of each pixel, and centering the treatment planning contour over the calculated center of mass.

29. The method of 21, wherein determining the contour difference between the high-fluence contour and the treatment planning contour comprises calculating a distance between the high-fluence contour and the treatment planning contour.

30. The method of claim 29, wherein calculating the distance comprises calculating distances between the high-fluence contour and the treatment planning contour in 2D and averaging the calculated distances.

31. The method of claim 29, wherein calculating the distance comprises calculating distances between the high-fluence contour and the treatment planning contour in 3D and averaging the calculated distances.

32. The method of 21, wherein determining the contour difference between the high-fluence contour and the treatment planning contour comprises determining an overlap region between a high-fluence region enclosed by the high-fluence contour and the treatment planning region enclosed by the treatment planning contour, and calculating a percentage overlap based on an area or volume of the overlap region and an area or volume enclosed by the treatment planning contour.

33. The method of claim 21, wherein the graphical representation further includes an anatomical image and the high-fluence contour and the treatment planning contour are overlaid on the anatomical image.

34. The method of claim 21, wherein the anatomical image comprises one or more of a CT image and an MRI image.

35. The method of claim 21, wherein the treatment planning contour is a contour on a planning fluence map.

36. A method for determining a delivered radiation dose, the method comprising:
emitting radiation beamlets from a radiation source of a radiotherapy system, wherein the emitted radiation beamlets are defined by openings of a multi-leaf collimator (MLC) located in front of the radiation source; and
generating a delivered radiation dose map using measurements acquired by sensors of the radiotherapy system and CT image data.

37. The method of claim 36, wherein the sensors of the radiotherapy system comprise a radiation imager, the acquired measurements comprise radiation imager data, and generating the delivered radiation dose map comprises back projecting radiation imager data using inverse attenuation and the CT image data from a plurality of firing positions of a therapeutic radiation source and patient platform positions.

38. The method of claim 37, wherein generating the delivered radiation dose map further comprises mapping radiation imager data to the CT image data on a row-by-row basis, deriving an attenuation coefficient for each pixel of the CT image data, calculating an amount of energy deposited to a pixel of the CT image data using the attenuation coefficient, generating an energy deposition map by combining the energy deposited to each pixel of the CT image data from all radiation beamlets emitted by the therapeutic radiation and firing positions, and calculating the delivered radiation dose from the energy deposition map.

39. The method of claim 38, wherein the acquired radiation imager measurements comprises radiation imager data acquired from multiple radiation source locations.

40. The method of claim 38, wherein calculating the amount of energy deposited to each pixel of a CT image data comprises calculating an unattenuated radiation imager signal for each pixel by reversing an attenuation of the radiation imager data according to the attenuation coefficient, and calculating the amount of energy deposited to each pixel of the CT image data based on the unattenuated radiation imager signal.

41. The method of claim 40, wherein reversing the attenuation of the radiation imager data comprises increasing an intensity of the radiation imager data by an increment determined by the inverse-square law, and adjusting the increased intensity by the attenuation coefficient
$$I ( x , y ) = I ( x , y ) \cdot {e}^{CT ( x , y ) \cdot Voxelsize / cos ( x , y )} .$$

42. The method of claim 36, wherein the sensors of the radiotherapy system comprise MLC leaf position sensors and a dose chamber disposed in a beam path of the radiation source, and wherein generating the delivered radiation dose map comprises forward projecting the MLC openings using MLC leaf position sensor data, dose chamber data, and the CT image data to calculate radiation deposition along the emitted radiation beamlets.

43. The method of claim 36, wherein emitting radiation beamlets comprises emitting radiation beamlets over a plurality of firing positions of the radiation source, and wherein generating the delivered radiation dose map comprises generating a delivered dose for the radiation beamlets emitted at each of the plurality of firing positions and summing the delivered doses over the plurality of firing positions.

44. The method of claim 43, wherein the radiotherapy system further comprises a patient couch movable to a plurality of beam station positions, and wherein emitting radiation beamlets further comprises emitting radiation beamlets over the plurality of beam station positions, and wherein generating the delivered radiation dose map comprises generating a beam station delivered dose for the radiation beamlets emitted at each of the plurality of patient couch positions and summing the beam station delivered dose over the plurality of beam station positions.

45. The method of claim 36, further comprising acquiring the CT image data on the same day as emitting the radiation beamlets.

46. The method of claim 36, further comprising generating a graphical representation that includes the delivered radiation dose map.

47. The method of claim 46, wherein the graphical representation further includes an anatomical image and the delivered radiation dose map is overlaid with the anatomical image.

48. The method of claim 46, wherein the graphical representation further includes a treatment planning contour and the delivered radiation dose map is overlaid with the treatment planning contour.

49. The method of claim 36, further comprising calculating a gamma [symbol] metric value for a pre-determined distance-to-agreement criterion (C_{DTA}) and a predetermined percent dose different criterion (C_{DD}) and determining with the calculated gamma metric value meets a pre-determined threshold, wherein calculating the gamma [symbol] metric value comprises calculating, for each pixel on the delivered radiation dose map, a distance-to-agreement value (DTA) to a planned radiation dose map and a percent dose difference (DD) to the planned radiation dose, where the gamma metric value is given by:
$$γ = \sqrt{{( \frac{DTA}{{C}_{DTA}} )}^{2} + {( \frac{DD}{{C}_{DD}} )}^{2}} .$$

50. The method of claim 36, further comprising calculating a delivered dose volume histogram (DVH) based on the radiation dose map.

51. The method of claim 50, further comprising comparing the delivered DVH with a planned DVH, and generating a graphical representation that includes the delivered DVH overlaid with the planned DVH.

52. The method of claim 51, wherein the planned DVH is a bounded DVH having a minimum DVH curve and a maximum DVH curve, and wherein comparing the delivered DVH with the planned DVH comprises determining a number of points on the delivered DVH that are within bounds defined the minimum DVH curve and the maximum DVH curve.

53. The method of claim 52, wherein the graphical representation includes visual indicia indicating whether the number of points on the delivered DVH meet or exceed a predetermined threshold.

54. The method of claim 52, further comprising calculating a percentage of points on the delivered DVH that are within bounds defined the minimum DVH curve and the maximum DVH curve and wherein the graphical representation includes visual indicia indicating whether the percentage of points meet or exceed a predetermined threshold.

55. A method for evaluating the quality of a fluence map and radiotherapy system, the method comprising:
delivering radiation according to a first radiation fluence map using a radiotherapy system comprising a linear accelerator (linac) and a MV detector located across from the linac;
recording imaging data from the MV detector during radiation delivery;
generating a second radiation fluence map using the recorded imaging data;
determining a fluence map difference by comparing the first radiation fluence map and the second radiation fluence map to determine a difference; and
if the fluence map difference is not within an acceptable range, generating a notification comprising an indication that the first radiation fluence map and the radiotherapy system are not of acceptable quality.

56. The method of claim 55, wherein the first radiation fluence map is a planned radiation fluence map intended for delivery to a target region.

57. The method of claim 55, wherein the first radiation fluence map is a quality assurance (QA) radiation fluence map that is not intended for delivery to a target region.
